SEQUENCE LISTING (29 sequences)

SEQ ID NO: 1 - DNA (genomic), 1260 bp; single-stranded, linear
  hypothetical: NO
  anti-sense: NO
gaattcccatcagcaatcttgcagattaatcgaactttcttcacactgaagcgtacagtatcgagaggggtaatcatgcgcgtcggtattccgaccgagaccaaaaacaacgaattccaattccgggtggccatcaccccggccggcgtcgcggaactaacccgtcgtggccatgaggtgctcatccaggcaggtgccggagagggctcggctatcaccgacgcggatttcaaggcggcaggcgcgcaactggtcggcaccgccgaccaggtgtgggccgacgctgatttattgctcaaggtcaaagaaccgatagcggcggaatacggccgcctgcgacacgggcagatcttgttcacgttcttgcatttggccgcgtcacgtgcttgcaccgatgcgttgttggattccggcaccacgtcaattgcctacgagaccgtccagaccgccgacggcgcactacccctgcttgccccgatgagcgaagtcgccggtcgactcgccgcccaggttggcgcttaccacctgatgcgaacccaagggggccgcggtgtgctgatgggcggggtgcccggcgtcgaaccggccgacgtcgtggtgatcggcgccggcaccgccggctacaacgcagcccgcatcgccaacggcatgggcgcgaccgttacggttctagacatcaacatcgacaaacttcggcaactcgacgccgagttctgcggccggatccacactcgctactcatcggcctacgagctcgagggtgccgtcaaacgtgccgacctggtgattggggccgtcctggtgccaggcgccaaggcacccaaattagtctcgaattcacttgtcgcgcatatgaaaccaggtgcggtactggtggatatagccatcgaccagggcggctgtttcgaaggctcacgaccgaccacctacgaccacccgacgttcgccgtgcacgacacgctgttttactgcgtggcgaacatgcccgcctcggtgccgaagacgtcgacctacgcgctgaccaacgcgacgatgccgtatgtgctcgagcttgccgaccatggctggcgggcggcgtgccggtcgaatccggcactagccaaaggtctttcgacgcacgaaggggcgttactgtccgaacgggtggccaccgacctgggggtgccgttcaccgagcccgccagcgtgctggcctgactctcggccgctcgttacgccgagcacacgtcgggagtaagggaagcgatgatgtcggccgcg

SEQ ID NO: 2 - DNA (genomic), 1245 bp; single-stranded, linear
  hypothetical: NO
  anti-sense: NO
atcttgcagattaatcgaactttcttcacactgaagcgtacagtatcgagaggggtaatcatgcgcgtcggtattccgaccgagaccaaaaacaacgaattccaattccgggtggccatcaccccggccggcgtcgcggaactaacccgtcgtggccatgaggtgctcatccaggcaggtgccggagagggctcggctatcaccgacgcggatttcaaggcggcaggcgcgcaactggtcggcaccgccgaccaggtgtgggccgacgctgatttattgctcaaggtcaaagaaccgatagcggcggaatacggccgcctgcgacacgggcagatcttgttcacgttcttgcatttggccgcgtcacgtgcttgcaccgatgcgttgttggattccggcaccacgtcaattgcctacgagaccgtccagaccgccgacggcgcactacccctgcttgccccgatgagcgaagtcgccggtcgactcgccgcccaggttggcgcttaccacctgatgcgaacccaagggggccgcggtgtgctgatgggcggggtgcccggcgtcgaaccggccgacgtcgtggtgatcggcgccggcaccgccggctacaacgcagcccgcatcgccaacggcatgggcgcgaccgttacggttctagacatcaacatcgacaaacttcggcaactcgacgccgagttctgcggccggatccacactcgctactcatcggcctacgagctcgagggtgccgtcaaacgtgccgacctggtgattggggccgtcctggtgccaggcgccaaggcacccaaattagtctcgaattcacttgtcgcgcatatgaaaccaggtgcggtactggtggatatagccatcgaccagggcggctgtttcgaaggctcacgaccgaccacctacgaccacccgacgttcgccgtgcacgacacgctgttttactgcgtggcgaacatgcccgcctcggtgccgaagacgtcgacctacgcgctgaccaacgcgacgatgccgtatgtgctcgagcttgccgaccatggctggcgggcggcgtgccggtcgaatccggcactagccaaaggtctttcgacgcacgaaggggcgttactgtccgaacgggtggccaccgacctgggggtgccgttcaccgagcccgccagcgtgctggcctgactctcggccgctcgttacgccgagcacacgtcgggagtaagggaagcgatgatgtcggccgcg

SEQ ID NO: 3 - DNA (genomic), 1235 bp; single-stranded, linear
  hypothetical: NO
  anti-sense: NO
atcttgcagattaatcgaactttcttcacactgaagcgtacagtatcgagaggggtaatcatgcgcgtcggtattccgaccgagaccaaaaacaacgaattccgggtggccatcaccccggccggcgtcgcggaactaacccgtcgtggccatgaggtgctcatccaggcaggtgccggagagggctcggctatcaccgacgcggatttcaaggcggcaggcgcgcaactggtcggcaccgccgaccaggtgtgggccgacgctgatttattgctcaaggtcaaagaaccgatagcggcggaatacggccgcctgcgacacgggcagatcttgttcacgttcttgcatttggccgcgtcacgtgcttgcaccgatgcgttgttggattccggcaccacgtcaattgcctacgagaccgtccagaccgccgacggcgcactacccctgcttgccccgatgagcgaagtcgccggtcgactcgccgcccaggttggcgcttaccacctgatgcgaacccaagggggccgcggtgtgctgatgggcggggtgcccggcgtcgaaccggccgacgtcgtggtgatcggcgccggcaccgccggctacaacgcagcccgcatcgccaacggcatgggcgcgaccgttacggttctagacatcaacatcgacaaacttcggcaactcgacgccgagttctgcggccggatccacactcgctactcatcggcctacgagctcgagggtgccgtcaaacgtgccgacctggtgattggggccgtcctggtgccaggcgccaaggcacccaaattagtctcgaattcacttgtcgcgcatatgaaaccaggtgcggtactggtggatatagccatcgaccagggcggctgtttcgaaggctcacgaccgaccacctacgaccacccgacgttcgccgtgcacgacacgctgttttactgcgtggcgaacatgcccgcctcggtgccgaagacgtcgacctacgcgctgaccaacgcgacgatgccgtatgtgctcgagcttgccgaccatggctggcgggcggcgtgccggtcgaatccggcactagccaaaggtctttcgacgcacgaaggggcgttactgtccgaacgggtggccaccgacctgggggtgccgttcaccgagcccgccagcgtgctggcctgactctcggccgctcgttacgccgagcacacntcgggagtaanggaagcgatgatgtcgnc

SEQ ID NO: 4 - DNA (genomic), 1237 bp; single-stranded, linear
  hypothetical: NO
  anti-sense: NO
atcttgcagattaatcgaactttcttcatactgaagcgtacagtatcgagaggggtaatcatgcgcgtcggtattccgaccgagaccaaaaacaacgaattccgggtggccatcaccccggccggcgtcgcggaactaacccgtcgtggccatgaggtgctcatccaggcaggtgccggagagggctcggctatcaccgacgcggatttcaaggcggcaggcgcgcaactggtcggcaccgccgaccaggtgtgggccgacgctgatttattgctcaaggtcaaagaaccgatagcggcggaatacggccgcctgcgacacgggcagatcttgttcacgttcttgcatttggccgcgtcacgtgcttgcaccgatgcgttgttggattccggcaccacgtcaattgcctacgagaccgtccagaccgccgacggcgcactacccctgcttgccccgatgagcgaagtcgccggtcgactcgccgcccaggttggcgcttaccacctgatgcgaacccaagggggccgcggtgtgctgatgggcggggtgcccggcgtcgaaccggccgacgtcgtggtgatcggcgccggcaccgccggctacaacgcagcccgcatcgccaacggcatgggcgcgaccgttacggttctagacatcaacatcgacaaacttcggcaactcgacgccgagttctgcggccggatccacactcgctactcatcggcctacgagctcgagggtgccgtcaaacgtgccgacctggtgattggggccgtcctggtgccaggcgccaaggcacccaaattagtctcgaattcacttgtcgcgcatatgaaaccaggtgcggtactggtggatatagccatcgaccagggcggctgtttcgaaggctcacgaccgaccacctacgaccacccgacgttcgccgtgcacgacacgctgttttactgcgtggcgaacatgcccgcctcggtgccgaagacgtcgacctacgcgctgaccaacgcgacgatgccgtatgtgctcgagcttgccgaccatggctggcgggcggcgtgccggtcgaatccggcactagccaaaggtctttcgacgcacgaaggggcgttactgtccgaacgggtggccaccgacctgggggtgccgttcaccgagcccgccagcgtgctggcctgactctcggccgctcgttacgccgagcacacgtcgggagtaagggaagcgatgatgtcggccg

SEQ ID NO: 5 - DNA (genomic), 1228 bp; single-stranded, linear
  hypothetical: NO
  anti-sense: NO
atcttgcagattaatcgaactttcttcatactgaagcgtacagtatcgagaggggtaatcatgcgcgtcggtattccgaccgagaccaaaaacaacgaattccgggtggccatcaccccggccggcgtcgcggaactaacccgtcgtggccatgaggtgctcatccaggcaggtgccggagagggctcggctatcaccgacgcggatttcaaggcggcaggcgcgcaactggtcggcaccgccgaccaggtgtgggccgacgctgatttattgctcaaggtcaaagaaccgatagcggcggaatacggccgcctgcgacacgggcagatcttgttcacgttcttgcatttggccgcgtcacgtgcttgcaccgatgcgttgttggattccggcaccacgtcaattgcctacgagaccgtccagaccgccgacggcgcactacccctgcttgccccgatgagcgaagtcgccggtcgactcgccgcccaggttggcgcttaccacctgatgcgaacccaagggggccgcggtgtgctgatgggcggggtgcccggcgtcgaaccggccgacgtcgtggtgatcggcgccggcaccgccggctacaacgcagcccgcatcgccaacggcatgggcgcgaccgttacggttctagacatcaacatcgacaaacttcggcaactcgacgccgagttctgcggccggatccacactcgctactcatcggcctacgagctcgagggtgccgtcaaacgtgccgacctggtgattggggccgtcctggtgccaggcgccaaggcacccaaattagtctcgaattcacttgtcgcgcatatgaaaccaggtgcggtactggtggatatagccatcgaccagggcggctgtttcgaaggctcacgaccgaccacctacgaccacccgacgttcgccgtgcacgacacgctgttttactgcgtggcgaacatgcccgcctcggtgccgaagacgtcgacctacgcgctgaccaacgcgacgatgccgtatgtgctcgagcttgccgaccatggctggcgggcggcgtgccggtcgaatccggcactagccaaaggtctttcgacgcacgaaggggcgttactgtccgaacgggtggccaccgacctgggggtgccgttcaccgagcccgccagcgtgctggcctgactctcggccgctcgttacgccgagcacacgtcgggagtaagggaagcgatga

SEQ ID NO: 6 - DNA (genomic), 1235 bp; single-stranded, linear
  hypothetical: NO
  anti-sense: NO
atcttgcagattaatcgaactttcttcacactgaagcgtacagtatcgagaggggtaatcatgcgcgtcggtattccgaccgagaccaaaaacaacgaattccgggtggccatcaccccggccggcgtcgcggaactaacccgtcgtggccatgaggtgctcatccaggcaggtgccggagagggctcggctatcaccgacgcggatttcaaggcggcaggcgcgcaactggtcggcaccgccgaccaggtgtgggccgacgctgatttattgctcaaggtcaaagaaccgatagcggcggaatacggccgcctgcgacacgggcgatcttgttcacgttcttgcatttggccgcgtcacgtgcttgcaccgatgcgttgttggattccggcaccacgtcaattgcctacgagaccgtccagaccgccgacggcgcactacccctgcttgccccgatgagcgaagtcgccggtcgactcgccgcccaggttggcgcttaccacctgatgcgaacccaagggggccgcggtgtgctgatgggcggggtgcccggcgtcgaaccggccgacgtcgtggtgatcggcgccggcaccgccggctacaacgcagcccgcatcgccaacggcatgggcgcgaccgttacggttctagacatcaacatcgacaaacttcggcaactcgacgccgagttctgcggccggatccacactcgctactcatcggcctacgagctcgagggtgccgtcaaacgtgccgacctggtgattggggccgtcctggtgccaggcgccaaggcacccaaattagtctcgaattcacttgtcgcgcatatgaaaccaggtgcggtactggtggatatagccatcgaccagggcggctgtttcgaaggctcacgaccgaccacctacgaccacccgacgttcgccgtgcacgacacgctgttttactgcgtggcgaacatgcccgcctcggtgccgaagacgtcgacctacgcgctgaccaacgcgacgatgccgtatgtgctcgagcttgccgaccatggctggcgggcggcgtgccggtcgaatccggcactagccaaaggtctttcgacgcacgaaggggcgttactgtccgaacgggtggccaccgacctgggggtgccgttcaccgagcccgccagcgtgctggcctgactctcggccgctcgttacgccgancacacgtcgggagtaagggaagcgatgatgtcggcc

SEQ ID NO: 7 - DNA (genomic), 1229 bp; single-stranded, linear
  hypothetical: NO
  anti-sense: NO
atcttgcagattaatcgaactttcttcacactgaagcgtacagtatcgagaggggtaatcatgcgcgtcggtattccgaccgagaccaaaaacaacgaattccgggtggccatcaccccggccggcgtcgcggaactaacccgtcgtggccatgaggtgctcatccaggcaggtgccggagagggctcggctatcaccgacgcggatttcaaggcggcaggcgcgcaactggtcggcaccgccgaccaggtgtgggccgacgctgatttattgctcaaggtcaaagaaccgatagcggcggaatacggccgcctgcgacacgggcgatcttgttcacgttcttgcatttggccgcgtcacgtgcttgcaccgatgcgttgttggattccggcaccacgtcaattgcctacgagaccgtccagaccgccgaaggcgcactacccctgcttgccccgatgagcgaagtcgccggtcgactcgccgcccaggttggcgcttaccacctgatgcgaacccaagggggccgcggtgtgctgatgggcggggtgcccggcgtcgaaccggccgacgtcgtggtgatcggcgccggcaccgccggctacaacgcagcccgcatcgccaacggcatgggcgcgaccgttacggttctagacatcaacatcgacaaacttcggcaactcgacgccgagttctgcggccggatccacactcgctactcatcggcctacgagctcgagggtgccgtcaaacgtgccgacctggtgattggggccgtcctggtgccaggcgccaaggcacccaaattagtctcgaattcacttgtcgcgcatatgaaaccaggtgcggtactggtggatatagccatcgaccagggcggctgtttcgaaggctcacgaccgaccacctacgaccacccgacgttcgccgtgcacgacacgctgttttactgcgtggcgaacatgcccgcctcggtgccgaagacgtcgacctacgcgctgaccaacgcgacgatgccgtatgtgctcgagcttgccgaccatggctggcgggcggcgtgccggtcgaatccggcactagccaaaggtctttcgacgcacgaaggggcgttactgtccgaacgggtggccaccgacctgggggtgccgttcaccgagcccgccagcgtgctggcctgactctcggccgctcgttacgccgagcacacgtcnggagtaagggaagcgatgatg

SEQ ID NO: 8 - DNA (genomic), 1235 bp; single-stranded, linear
  hypothetical: NO
  anti-sense: NO
atcttgcagattaatcgaactttcttcacactgaagcgtacagtatcgagaggggtaatcatgcgcgtcggtattccgaccgagaccaaaaacaacgaattccgggtggccatcaccccggccggcgtcgcggaactaacccgtcgtggccatgaggtgctcatccaggcaggtgccggagagggctcggctatcaccgacgcggatttcaaggcggcaggcgcgcaactggtcggcaccgccgaccaggtgtgggccgacgctgatttattgctcaaggtcaaagaaccgatagcggcggaatacggccgcctgcgacacgggcgatcttgttcacgttcttgcatttggccgcgtcacgtgcttgcaccgatgcgttgttggattccggcaccacgtcaattgcctacgagaccgtccagaccgccgaaggcgcactacccctgcttgccccgatgagcgaagtcgccggtcgactcgccgcccaggttggcgcttaccacctgatgcgaacccaagggggccgcggtgtgctgatgggcggggtgcccggcgtcgaaccggccgacgtcgtggtgatcggcgccggcaccgccggctacaacgcagcccgcatcgccaacggcatgggcgcgaccgttacggttctagacatcaacatcgacaaacttcggcaactcgacgccgagttctgcggccggatccacactcgctactcatcggcctacgagctcgagggtgccgtcaaacgtgccgacctggtgattggggccgtcctggtgccaggcgccaaggcacccaaattagtctcgaattcacttgtcgcgcatatgaaaccaggtgcggtactggtggatatagccatcgaccagggcggctgtttcgaaggctcacgaccgaccacctacgaccacccgacgttcgccgtgcacgacacgctgttttactgcgtggcgaacatgcccgcctcggtgccgaagacgtcgacctacgcgctgaccaacgcgacgatgccgtatgtgctcgagcttgccgaccatggctggcgggcggcgtgccggtcgaatccggcactagccaaaggtctttcgacgcacgaaggggcgttactgtccgaacgggtggccaccgacctgggggtgccgttcaccgagcccgccagcgtgctggcctgactctcggccgctcgttacgccgagcacacgtcgggagtaagggaagcgatgatgtcggcc

SEQ ID NO: 9 - DNA (genomic), 1209 bp; single-stranded, linear
  hypothetical: NO
  anti-sense: NO
atcttgcagattaatcgaactttcttcacactgaagcgtacagtatcgagaggggtaatcatgcgcgtcggtattccgaccgagaccaaaaacaacgaattccgggtggccatcaccccggccggcgtcgcggaactaacccgtcgtggccatgaggtgctcatccaggcaggtgccggagagggctcggctatcaccgacgcggatttcaaggcggcaggcgcgcaactggtcggcaccgccgaccaggtgtgggccgacgctgatttattgctcaaggtcaaagaaccgatagcggcggaatacggccgcctgcgacacgggcagatcttgttcacgttcttgcatttggccgcgtcacgtgcttgcaccgatgcgttgttggattccggcaccacgtcaattgcctacgagaccgtccagaccgccgacggcgcactacccctgcttgccccgatgagcgaagtcgccggtcgactcgccgcccaggttggcgcttaccacctgatgcgaacccaagggggccgcggtgtgctgatgggcggggtgcccggcgtcgaaccggccgacgtcgtggtgatcggcgccggcaccgccggctacaacgcagcccgcatcgccaacggcatgggcgcgaccgttacggttctagacatcaacatcgacaaacttcggcaactcgacgccgagttctgcggccggatccacactcgctactcatcggcctacgagctcgagggtgccgtcaaacgtgccgacctggtgattggggccgtcctggtgccaggcgccaaggcacccaaattagtctcgaattcacttgtcgcgcatatgaaaccaggtgcggtactggtggatatagccatcgaccagggcggctgtttcgaaggctcacgaccgaccacctacgaccacccgacgttcgccgtgcacgacacgctgttttactgcgtggcgaacatgcccgcctcggtgccgaagacgtcgacctacgcgctgaccaacgcgacgatgccgtatgtgctcgagcttgccgaccatggctggcgggcggcgtgccggtcgaatccggcactagccaaaggtctttcgacgcacgaaggggcgttactgtccgaacgggtggccaccgacctgggggtgccgttcaccgagcccgccagcgtgctggcctgactctcggccgctcgttacgccgagcncacgtcg

SEQ ID NO: 10 - DNA (genomic), 1236 bp; single-stranded, linear
  hypothetical: NO
  anti-sense: NO
atcttgcagattaatcgaactttcttcacactgaagcgtacagtatcgagaggggtaatcatgcgcgtcggtattccgaccgagaccaaaaacaacgaattccgggtggccatcaccccggccggcgtcgcggaactaacccgtcgtggccatgaggtgctcatccaggcaggtgccggagagggctcggctatcaccgacgcggatttcaaggcggcaggcgcgcaactggtcggcaccgccgaccaggtgtgggccgacgctgatttattgctcaaggtcaaagaaccgatagcggcggaatacggccgcctgcgacacgggcagatcttgttcacgttcttgcatttggccgcgtcacgtgcttgcaccgatgcgttgttggattccggcaccacgtcaattgcctacgagaccgtccagaccgccgacggcgcactacccctgcttgccccgatgagcgaagtcgccggtcgactcgccgcccaggttggcgcttaccacctgatgcgaacccaagggggccgcggtgtgctgatgggcggggtgcccggcgtcgaaccggccgacgtcgtggtgatcggcgccggcaccgccggctacaacgcagcccgcatcgccaacggcatgggcgcgaccgttacggttctagacatcaacatcgacaaacttcggcaactcgacgccgagttctgcggccggatccacactcgctactcatcggcctacgagctcgagggtgccgtcaaacgtgccgacctggtgattggggccgtcctggtgccaggcgccaaggcacccaaattagtctcgaattcacttgtcgcgcatatgaaaccaggtgcggtactggtggatatagccatcgaccagggcggctgtttcgaaggctcacgaccgaccacctacgaccacccgacgttcgccgtgcacgacacgctgttttactgcgtggcgaacatgcccgcctcggtgccgaagacgtcgacctacgcgctgaccaacgcgacgatgccgtatgtgctcgagcttgccgaccatggctggcgggcggcgtgccggtcgaatccggcactagccaaaggtctttcgacgcacgaaggggcgttactgtccgaacgggtggccaccgacctgggggtgccgttcaccgagcccgccagcgtgctggcctgactctcggccgctcgttacgccgagcacacgtcgggagtaagggaagcgatgatgtcggcc

SEQ ID NO: 11 - DNA (other nucleic acid), 18 bp; single-stranded, linear
  hypothetical: NO
  anti-sense: NO
atgcgcgtcggtattccg

SEQ ID NO: 12 - DNA (other nucleic acid), 20 bp; single-stranded, linear
  hypothetical: NO
  anti-sense: NO
gcgcgtcggtattccgaccg

SEQ ID NO: 13 - DNA (other nucleic acid), 18 bp; single-stranded, linear
  hypothetical: NO
  anti-sense: NO
gagaccaaaaacaacgaa

SEQ ID NO: 14 - DNA (other nucleic acid), 25 bp; single-stranded, linear
  hypothetical: NO
  anti-sense: NO
gaattcccatcagcaatcttgcaga

SEQ ID NO: 15 - DNA (other nucleic acid), 18 bp; single-stranded, linear
  hypothetical: NO
  anti-sense: NO
gccccgatgagcgaagtc

SEQ ID NO: 16 - DNA (other nucleic acid), 17 bp; single-stranded, linear
  hypothetical: NO
  anti-sense: NO
ggggccgtcctggtgcc

SEQ ID NO: 17 - DNA (other nucleic acid), 21 bp; single-stranded, linear
  hypothetical: NO
  anti-sense: NO
gacgtcgacctacgcgctgac

SEQ ID NO: 18 - DNA (other nucleic acid), 18 bp; single-stranded, linear
  hypothetical: NO
  anti-sense: NO
ctcggtgaacggcacccc

SEQ ID NO: 19 - DNA (other nucleic acid), 18 bp; single-stranded, linear
  hypothetical: NO
  anti-sense: NO
ggccagcacgctggcggg

SEQ ID NO: 20 - DNA (other nucleic acid), 18 bp; single-stranded, linear
  hypothetical: NO
  anti-sense: NO
cacccgttcggacagtaa

SEQ ID NO: 21 - DNA (other nucleic acid), 18 bp; single-stranded, linear
  hypothetical: NO
  anti-sense: NO
cgcggccgacatcatcgc

SEQ ID NO: 22 - DNA (other nucleic acid), 20 bp; single-stranded, linear
  hypothetical: NO
  anti-sense: NO
ggccgacatcatcgcttccc

SEQ ID NO: 23 - DNA (other nucleic acid), 24 bp; single-stranded, linear
  hypothetical: NO
  anti-sense: NO
cgagactaatttgggtgccttggc

SEQ ID NO: 24 - DNA (other nucleic acid), 16 bp; single-stranded, linear
  hypothetical: NO
  anti-sense: NO
atttgggtgccttggc

SEQ ID NO: 25 - DNA (other nucleic acid), 18 bp; single-stranded, linear
  hypothetical: NO
  anti-sense: NO
ggcggcgagtcgaccggc

SEQ ID NO: 26 - DNA (genomic), 21 bp; single-stranded, linear
  hypothetical: NO
  anti-sense: NO
aacgaattccaattccgggtg

SEQ ID NO: 27 - protein, 7 aa
  hypothetical: NO
  anti-sense: NO
NEFQFRV

SEQ ID NO: 28 - DNA (genomic), 15 bp; single-stranded, linear
  hypothetical: NO
  anti-sense: NO
aacgaattccgggtg

SEQ ID NO: 29 - protein, 5 aa
  hypothetical: NO
  anti-sense: NO
NEFRV

(19) United States
(12) Patent Application Publication
(10) Pub. No.: US 2003/0162171 A1
(43) Pub. Date: Aug. 28, 2003

(54) TEST KIT FOR TUBERCULOSIS DIAGNOSIS BY DETERMINING ALANINE DEHYDROGENASE

(76) Inventors: LEOPOLD FLOHE, WOLFENBUTTEL (DE); MAHAVIR SINGH, BRAUNSCHWEIG (DE); BERND HUTTER, BRAUNSCHWEIG (DE); AREND KOLK, BRAUNSCHWEIG (DE)

Correspondence Address: MARSHALL O'TOOLE GERSTEIN, MURRAY & BORUN, 6300 SEARS TOWER, 233 SOUTH WACKER DRIVE, CHICAGO, IL, 606066402

(21) Appl. No.: 09/362,485

(22) Filed: Jul. 28, 1999

Related U.S. Application Data
(63) Continuation of application No. PCT/EP98/00483, filed on Jan. 29, 1998.

(30) Foreign Application Priority Data
Jan. 29, 1997 (EP) 97 101 338.8

(51) Int. Cl. C12Q 1/68; C07H 21/04; C12Q 1/26

(52) U.S. Cl. 435/6; 435/25; 536/23.2

(57) ABSTRACT

Tuberculosis is an infectious disease which kills more than three million people every year. Although both a vaccine and various methods of diagnosis and treatment are available, the efficacy of these measures is in urgent need of improvement given that the number of new cases is once again on the increase. Research focuses, among other things, on the characterization of antigens secreted in the early stages of the infection as they constitute the first point of contact of the immune system with the pathogen. The 40 KD-antigen described herein is present in vivo as a hexamer and, despite its high molecular weight and lack of a signal sequence, is present extracellularly after only a few days of growth. Functionally, it is an L-alanine dehydrogenase and reacts with the monoclonal antibody HBT-10 directed against this protein. HBT-10 was the first known antibody specific to a protein of M. tuberculosis which did not cross-react with the vaccine strain M. bovis BCG.

Isolated lambda gt11 clones containing the complete AlaDH coding DNA of M. tuberculosis or parts thereof are known from Anderson et al. (1992). The isolated mycobacterial AlaDH insert from lambda AA67 was used as the hybridisation probe in that work.

### 1 PROBLEM AND INVENTION

The 40 kD antigen with which this work is concerned is in many respects an interesting subject for detailed studies.

The antigen had already been cloned into an expression vector for Escherichia coli (Konrad & Singh, unpublished). The expression and purification of the recombinant protein was therefore to be optimised. Using a homogeneous protein fraction, the crucial biochemical parameters of the enzyme were then to be determined. Previous experience has shown that it is possible to infer the physiological function of an enzyme from such data. The question that this posed was whether the hypothetical function of the enzyme in cell wall biosynthesis could be confirmed or disproved. If disproved, other possible functions were to be elicited.

In addition, the biochemistry may provide starting points for specific influencing of the enzyme in vivo. In that context, the physiological function is once again the key point for all efforts towards that end. If the antigen were to play an essential role for the bacterium, then attempts aimed specifically at switching off the gene or the protein might provide possibilities for preventing the growth of the tuberculosis pathogen at a defined point. The protein would then be an ideal drug target. If, in addition, as postulated (Delforge et al., 1993), the 40 kD antigen were to represent a virulence factor, influence might be brought to bear on the natural virulence of the bacterium by such endeavours. That aspect also was to be verified, therefore, by various tests.

The ability to discriminate the strains M. tuberculosis and M. bovis BCG by means of the mAb HBT-10 makes it possible to develop methods of distinguishing an infection from a vaccination. That is not possible with the conventional screening methods, the PPD and the Mantoux test (Bass Jr. et al., 1990; Huebner et al., 1993). By analysis of the distribution of the gene or the gene product the foundation was to be laid for the development of an economical method for such a test. In addition, whether the presence of a functional enzyme correlates with any other parameters was to be investigated. Particular importance was attached to correlations between taxonomy and virulence. Certain natural modes of life or the entry into certain growth phases might also be related to alanine dehydrogenase. Fundamental answers were to be sought to those questions.

The invention relates to an enzymatic test kit for the diagnosis of tuberculosis and other mycobacterial infections in humans and animals by determination of the activity of alanine dehydrogenase (E.C. 1.4.1.1), comprising L-alanine, nicotinamide adenine dinucleotide (oxidised form; NAD⁺), phenazine methosulphate (PMS) and nitroblue tetrazolium chloride (NBT).

The invention further relates to a method of diagnosing tuberculosis and other mycobacterial infections of humans and animals, characterised in that the activity of alanine dehydrogenase (E.C. 1.4.1.1.) is measured with an enzymatic test kit according to claim 1.

The method according to the invention may be characterised in that

(i) possible tuberculosis pathogens, such as M. tuberculosis, are isolated,

(ii) a crude cell extract is made,

(iii) the extract is incubated in solution and

(iv) the absorption is measured.

The method according to the invention may further be characterised in that clinical samples, such as body fluids, are subjected directly to tuberculosis diagnosis and the alanine dehydrogenase activity is measured.

The method according to the invention may further be characterised in that cells, strains and/or species of disease-causing organisms (mycobacteria) are differentiated from non-virulent cells and strains.

The method according to the invention may further be characterised in that cells, strains and/or species of disease-causing organisms of the M. tuberculosis complex are identified and differentiated.

The method according to the invention may further be characterised in that the method is carried out in the presence of substances that inhibit tuberculosis and other mycobacterial infections of humans and animals and those inhibiting substances are optionally recovered.

The method according to the invention may further be characterised in that it is carried out

(i) to control epidemics and/or

(ii) after vaccinations (vaccination follow-up) in humans and animals.

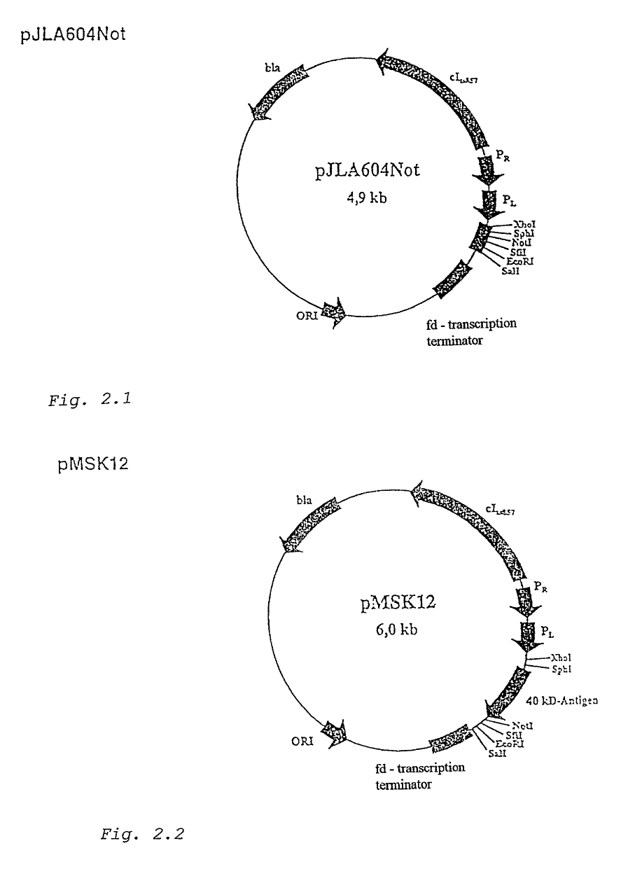

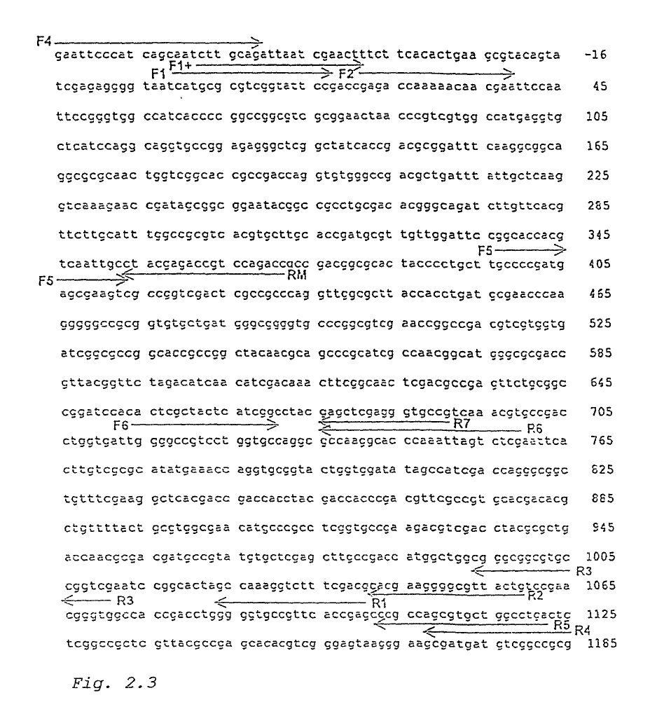

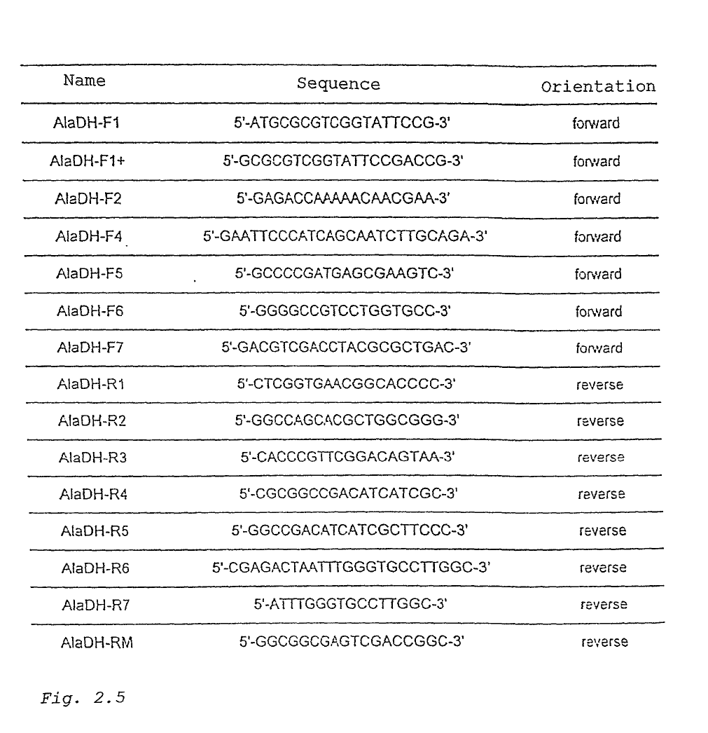

The invention further relates to a DNA sequence selected from the following group or other partial sequences of the alanine dehydrogenase gene of M. tuberculosis (FIG. 2.5):

| | | Orienta- |
|---|---|---|
| Name | Sequence | tion |
| AlaDH-F1 | 5′-ATGCGCGTCGGTATTCCG-3′ | forward |
| AlaDH-F1+ | 5′-GCGCGTCGGTATTCCGACCG-3′ | forward |
| AlaDH-F2 | 5′-GAGACCAAAACAACGAA-3′ | forward |
| AlaDH-F4 | 5′-GAATTCCCATCAGCAATCTTGCAGA-3′ | forward |
| AlaDH-F5 | 5′-GCCCCGATGAGCGAAGTC-3′ | forward |
| AlaDH-F6 | 5′-GGGGCCGTCCTGGTGCC-3′ | forward |
| AlaDH-F7 | 5′-GACGTCGACCTACGCGCTGAC-3′ | forward |
| AlaDH-R1 | 5′-CTCGGTGAACGGCACCCC-3′ | reverse |
| AlaDH-R2 | 5′-GGCCAGCACGCTGGCGGG-3′ | reverse |
| AlaDH-R3 | 5′-CACCCGTTCGGACAGTAA-3′ | reverse |
| AlaDH-R4 | 5′-CGCGGCCGACATCATCGC-3′ | reverse |
| AlaDH-R5 | 5′-GGCCGACATCATCGCTTCCC-3′ | reverse |
| AlaDH-RG | 5′-CGAGACTAATTTGGGTGCCTTGGC-3′ | reverse |
| AlaDH-R7 | 5′-ATTTGGGTGCCTTGGC-3′ | reverse |
| AlaDH-RM | 5′-GGCGGCGAGTCGACCGGC-3′ | reverse |

and partial sequences thereof and sequences that are hybridisable therewith preferably at a temperature of at least 20° C. and especially at a concentration of 1M NaCl and a temperature of at least 25° C., for the diagnosis of tuberculosis and other mycobacterial infections in humans and animals.

The use according to the invention of a DNA sequence may be envisaged for the diagnosis of tuberculosis and other mycobacterial infections in humans and animals.

The invention further relates to a method that is characterised in that a DNA sequence according to the invention is used

(i) for hybridisation,

(ii) for culture confirmation of isolated strains and/or

(iii) for chromosomal fingerprinting, and cells, strains and/or types of mycobacteria are determined and differentiated and/or are used for the diagnosis of mycobacterial infections.

The method according to the invention may be characterised in that cells, strains and/or species of virulent mycobacteria are differentiated from non-virulent cells, strains and/or species.

The method according to the invention may further be characterised in that cells, strains and/or species of the M. tuberculosis complex and other mycobacteria

(i) are isolated,

(ii) crude or purified genomic DNA or RNA is recovered,

(iii) a fragment that is identical or virtually identical to the sequence of the alanine dehydrogenase gene of M. tuberculosis (FIG. 2.3) is identified, preferably by amplification using a DNA sequence according to the invention as a primer sequence, after which digestion is carried out with a restriction enzyme, especially Bg1II, and gel electrophoresis of the digested amplified DNA is carried out and/or the DNA sequence of the amplified DNA is determined.

The method according to the invention may further be characterised in that a clinical sample is used directly and diagnosed for tuberculosis in humans and animals.

The method according to the invention may further be characterised in that the method is carried out in the presence of substances that inhibit tuberculosis or mycobacterial infections of humans and animals and inhibiting substances determined are recovered or made.

The method according to the invention may further be characterised in that it is used

(i) in antimycobacterial chemotherapy,

(ii) in the control of epidemics and/or

(iii) after vaccinations (vaccination follow-up) in humans and animals.

2 Materials and Methods

2.1 Living Material

2.1.1 Bacteria

2.1.1.1 E. coli Strains

The strain Escherichia coli was used to optimise the expression of the recombinant 40 kD antigen (Tab. 2.1). In addition, mycobacterial antigens already cloned therein were over-produced (Tab. 2.2).

TABLE 2.1
Expression strains used and their relevant properties

| strain | genotype and relevant phenotype | origin/reference |
|---|---|---|
| E. coli CAG 629 | lac(am) pho(am) trp(am) supC^{ts} rpsL mal(am) lon | C. Gross |
| | htpRI65-Tn10(Tet^{R}) | |
| E. coli DH5α | supE44 ΔlacUI69(φ80 lacZ ΔM15) hsdRI7 recA1 | Hanahan (1983) |
| | endA1 gyrA96 thi-1 relA1 | |
| E. coli TG2 | supE hsdΔ5 thiΔ(lac-proAB) Δ(srl-recA)306::Tn10(Tet^{R}) | Sambrook et al. (1989) |
| | F′(traD36 proA⁺ lacI^{q} lacZM 15) | |
| E. coli SURE | hsdR mcrA mcrB mvr endA supE44 thi-1 λ-gyrA96 | Stratagene |
| | relA1 lac recB recJ sbcC umuC uvrC (F′ proAB lacI^{q}Z | |
| | ΔM15 Tn10(Tet^{R})) | |
| E. coli BL 321 | rnc105 nadB⁺ purI⁺ | Studier (1975) |
| E. coli N 4830 | su° his ilv galKΔ8 ΔchID-pgl (λ ΔBam N⁺ cIₜₛ₈₅₇ ΔHI) | Gottesman et al. |
| | | (1980) |
| E. coli 538 | genotype unknown | Bayer AG |

TABLE 2.2
Producers of mycobacterial antigens and
characteristics thereof
The antigen produced by the respective strain is indicated.
The last two columns give the growing conditions.

| Strain | origin/reference(s) | product | antibiotics | induction |
|---|---|---|---|---|
| E. coli BL21 (pKAM1301) | J. van Embden | GST-36 kD antigen, | Ap | IPTG |
| | | M. leprae | | |
| E. coli BL21/plys 5 | J. van Embden | 70 kD antigen, | Ap + Cm | IPTG |
| (pKAM3601) | | M. leprae | | |
| E. coli CAG629 (pMS9-2) | Singh et al. (1992) | 38 kD antigen, | Ap | heat |
| | | M. tuberculosis | | |
| E. coli CAG629 (pMS14-1) | Cherayil & Young (1988) | 28 kD antigen, | Ap | heat |
| | Dale & Patki (1990) | M. leprae | | |
| | Singh et al. (unpublished) | | | |
| E. coli M15 (pHISK16 + | Verbon et al. (1992) | 16 kD antigen, | Ap | IPTG |
| pREP4) | Vordermeier et al. (1993) | M. tuberculosis | | |
| E. coli M1697 | V. Mehra | His-30 kD antigen, | Ap + Km | IPTG |
| | | M. tuberculosis | | |
| E. coli M1698 | V.Mehra | His-30 kD antigen, | Ap + Km | IPTG |
| | | M. leprae | | |
| E. coli POP (pKAM2101) | J. van Embden | 70 kD antigen, | Ap | heat |
| | | M. tuberculosis | | |
| E. coli POP (pRIB1300) | Thole et al. (1987) | 65 kD antigen, | Ap | heat |
| | van Eden et al. (1988) | M. bovis BCG | | |
| E. coli POP (pZW1003) | Mehra et al. (1986) | 65 kD antigen, | Ap | heat |
| | van der Zee et al. | M. leprae | | |
| | (unpublished) | | | |
| E. coli TB1 (pKAM1101) | di Guan et al. (1987) | MBP-38 kD antigen, | Ap | heat |
| | Maina et al. (1988) | M. leprae | | |
| | Thole et al. (1990) | | | |
| E. coli TB1 (pKAM1401) | J. van Embden | MBP-2nd 65 kD antigen, | Ap | heat |
| | | M. leprae | | |
| E. coli TB21-8/2 | Khanolar-Young et al. | MBP-10 kD antigen, | Ap | IPTG |
| | (1992) | M. tuberculosis | | |
| | Mehra et al. (1992) | | | |
| E. coli TG2-50/55 Sal | C. Espitia; M. Singh | 50/55 kD, large frag., | Ap | IPTG |
| large | | M. tuberculosis | | |

2.1.1.2 Mycobacterial Strains

TABLE 2.3
Mycobacteria used and the origin thereof

| strain | abbreviation | exact name, origin |
|---|---|---|
| M. africanum 1 | Afr1 | M. africanum No. 5544, RIV |
| M. asiaticum 1 | Asi1 | M. asiaticum 3250, Portaals |
| M. avium 1 | Avi1 | M. avium Myc 3875, Serotype 2, RIV |
| M. bovis 3 | Bov3 | M. bovis No. 8316, RIV |
| M. bovis BCG 2 | BCG2 | M. bovis Copenhagen, Seruminstitut Copenhagen |
| M. bovis BCG 4 | BCG4 | M. bovis BCG P₃, RIV |
| M. chelonae 7 | Che7 | M. chelonei 1490, P. Dirven |
| M. flavescens 1 | Fla1 | M. flavescens ATCC 14474, RIV |
| M. fortuitum 11 | For11 | M. fortuitum ATCC 6841, RIV |
| M. gastri 1 | Gas1 | M. gastri ATCC 25220, RIV |
| M. gordonae 3 | Gor3 | M. gordonae 8690, Portaals |
| M. intracellulare 1 | Int1 | M. intracellulare 6997, ATCC 15985, Portaals |
| M. intracellulare 5 | Int5 | M. intracellulare IWG MT3, RIV |
| M. kansasii 1 | Kan1 | M. kansasii Myc 1012, RIV |
| M. lufu 1 | Luf1 | M. lufu 219, RIV |
| M. marinum 3 | Mar3 | M. marinum L66, Portaals |
| M. microti 1 | Mic1 | M. microti No. 1278, Portaals |
| M. nonchromogenium 1 | Non1 | M. nonchromogenium ATCC 25145, RIV |
| M. parafortuitum 1 | Paf1 | M. parafortuitum No. 6999, Portaals |
| M. peregrinum 1 | Per1 | M. peregrinum, Patient Bakker, TB6849, |
| | | Antonie Ziekenhius |
| M. phlei 1 | Phl1 | M. phlei 258 (Ph), Portaals |
| M. phlei 4 | Phl4 | M. phlei Weybridge R82, Tony Eger |
| M. scrofulaceum 1 | Scr1 | M. scrofulaceum Myc 3442, RIV |
| M. scrofulaceum 8 | Scr8 | M. scrofulaceum Myc 6672, RIV |
| M. simiae 1 | Sim1 | M. simiae 784, Tony Eger |
| M. smegmatis 1 | Sme1 | M. smegmatis ATCC 14460, RIV |
| M. smegmatis 3 | Sme3 | M. smegmatis 8070, Portaals |
| M. terrae 2 | Ter2 | M. terrae, RIV |
| M. thermoresistibile 1 | The1 | M. thermoresistibile No. 7001, Portaals |
| M. triviale 1 | Tri1 | M. triviale 8067, Portaals |
| M. tuberculosis H37Rᵥ | H37Rᵥ | M. tuberculosis H37Rᵥ , RIV |
| M. tuberculosis H37Rₐ | H37Rₐ | M. tuberculosis H37Rₐ , No. 19629, RIV |
| M. tuberculosis 1 | Tub1 | M. tuberculosis 4514, RIV |
| M. tuberculosis 49 | Tub49 | M. tuberculosis C₃, Sang-Hae Cho, South Korea |
| M. tuberculosis 60 | Tub6O | M. tuberculosis S₂, Sang-Hae Cho, South Korea |
| M. tuberculosis 118 | Tub118 | M. tuberculosis Myc 16293, Hannoufi |
| M. tuberculosis 130 | Tub130 | M. tuberculosis, Patient yy, barcode 3.1265, Dr. Bijlmer, |
| | | The Hague |
| M. tuberculosis 132 | Tub132 | M. tuberculosis Myc 16770, RIV |
| M. tuberculosis 145 | Tub145 | M. tuberculosis 416138N, Patient N. Wielaart, Reg. No. |
| | | 7.796.267, WKZ, Utrecht |
| M. tuberculosis 146 | Tub146 | M. tuberculosis, Abdi Hussein |
| M. tuberculosis 163 | Tub163 | M. tuberculosis 925, patient isolate No. 32, |
| | | INH > 1, Str^{R}, Rif^{S}, Eth^{S} |
| M. ulcerus 1 | Ulc1 | M. ulcerus 932, Portaals |
| M. vaccae 3 | Vac3 | M. vaccae ATCC 25950, RIV |
| M. xenopi 7 | Xen7 | M. xenopi code 132, Patient Alois Necas, |
| | | H. Kristanpul, Prague |

2.1.1.3 Other Strains of Bacteria

TABLE 2.4
Other strains of bacteria used

| strain | origin |
|---|---|
| Listeria monocytogenes EGB | Andreas Lignau |
| Listeria innocua | Andreas Lignau |
| Nocardia asteroides 702774 | Juul Bruins |
| Rhodococcus equi No. 10P388 | VMDC, Utrecht |

2.1.2 Cell Culture

The mouse macrophage cell line J774 was used. That cell line was originally established from a tumour of a female BALB/c mouse (Ralph & Nakoinz, 1975). J774 is used for phagocytosis assays, for the production of IL-1 and for a wide range of biochemical investigations. It has receptors for immunoglobulins and complement. J774 furthermore produces lysozyme in large quantities and secretes IL-1 constitutively (Ralph & Nakoinz, 1976; Snyderman et al., 1977). Bacteria are taken up by phagocytosis. Direct cytolysis of foreign organisms is relatively rare.

2.2 Nucleic Acids

2.2.1 Plasmids

Plasmid pJLA604Not and its Relevant Functional Segments

This 4.9 kb plasmid, a derivative of pJLA 604 (Schauder et al., 1987), was used as an expression vector (FIG. 2.1). The plasmid pJLA604Not (Konrad & Singh, unpublished) differs from pJLA604 in that the NdeI cleavage site has been removed and, in its place, a NotI cleavage site has been incorporated. The reading frame of the translation begins with the ATG codon of the SphI cleavage site. Transcription starts at the lambda promoters P_{R} and P_{L}, but is effectively repressed at temperatures of 28-30° C. by the cI_{tS857}-gene product. Induction is achieved by increasing the temperature to 42° C. At that temperature, the temperature-sensitive lambda repressor becomes inactive and is no longer able to repress the transcription. Transcription ends at the fd terminator. In addition, the vector possesses the atpE translation initiation region (TIR) of E. coli. This segment is very useful for initiating translation since it has secondary structures that cause only little interference and consequently guarantees a high expression rate (McCarthy et al., 1986). As a selection marker, the plasmid has at its disposal the β-lactamase gene that codes for ampicillin resistance.

As a negative control plasmid, pJLA603 also was used, which is identical to pJLA604 apart from a few bases in the cloning site.

Plasmid pMSKS12 and its Relevant Functional Segments

This is a derivative of the plasmid pJLA604Not, in which the 40 kD antigen of Mycobacterium tuberculosis has been cloned between the SphI and the NotI cleavage sites (FIG. 2.2; Konrad & Singh, unpublished).

2.2.2 Oligonucleotides

All of the oligonucleotides (Tab. 2.5) were made by Frau Astrid Hans (GBF, Braunschweig) on a 394 DNA/RNA Synthesizer (Applied Biosystems). The oligonucleotides were purified with an Oligonucleotide Purification Cartridge (Applied Biosystems).

Tab. 2.5 (½): Oligonucleotides used

| | | orienta- |
|---|---|---|
| name | sequence | tion |
| AlaDH-F1 | 5′-ATGCGCGTCGGTATTCCG-3′ | forward |
| AlaDH-F1+ | 5′-GCGCGTCGGTATTCCGACCG-3′ | forward |
| AlaDH-F2 | 5′-GAGACCAAAAACAACGAA-3′ | forward |
| AlaDH-F4 | 5′-GAATTCCCATCAGCAATCTTGCAGA-3′ | forward |
| AlaDH-F5 | 5′-GCCCCGATGAGCGAAGTC-3′ | forward |
| AlaDH-F6 | 5′-GGGGCCGTCCTGGTGCC-3′ | forward |
| AlaDH-F7 | 5′-GACGTCGACCTACGCGCTGAC-3′ | forward |
| AlaDH-R1 | 5′-CTCGGTGAACGGCACCCC-3′ | reverse |
| AlaDH-R2 | 5′-GGCCAGCACGCTGGCGGG-3′ | reverse |
| AlaDH-R3 | 5′-CACCCGTTCGGACAGTAA-3′ | reverse |
| AlaDH-R4 | 5′-CGCGGCCGACATCATCGC-3′ | reverse |
| AlaDH-R5 | 5′-GGCCGACATCATCGCTTCCC-3′ | reverse |
| AlaDH-R6 | 5′-CGAGACTAATTTGGGTGCCTTGGC-3+ | reverse |
| AlaDH-R7 | 5′-ATTTGGGTGCCTTGGC-3′ | reverse |
| AlaDH-PM | 5′-GGCGGCGAGTCGACCGGC-3′ | reverse |

The location of the oligos on the AlaDH gene is shown schematically in FIG. 2.3. (The oligos used and their position on the AlaDH gene)

2.3 Formulations

All of the solutions described in this section were prepared very largely in accordance with Sambrook et al. (1989).

2.3.1 Nutrient Media

LB

10 g of Bacto Tryptone (Difco), 5 g of Bacto yeast extract (Difco), 10 g of NaCl ad 1000 ml of H₂O, pH 7.0, autoclaving

IB

12 g of Bacto Tryptone (Difco), 24 g of Bacto yeast extract (Difco), 4 ml of glycerol (87%), 2.31 g of KH₂PO₄, 12.54 g of K₂HPO₄ ad 1000 ml of H₂O, the phosphate solutions are separated from the other components, autoclaved and subsequently admixed

SOC

2% Bacto Tryptone (Difco), 0.5% Bacto yeast extract (Difco), 10 mM NaCl, 2.4 mM KCl, 10 mM MgCl₂, 10 mM MgSO₄, 20 mM glucose ad 1000 ml of H₂O, pH 7.0, the glucose is separated from the other components, autoclaved and subsequently added

Löwenstein

Ready-for-use Coletsos Ossein slant agar tubes (Sanofi Diagnostics Pasteur) were used.

Solid Media

To produce plates (90 mm, Greiner) of the nutrient media described above, 1.5% agar was admixed with the relevant formulation.

Antibiotics

Antibiotics were added from stock solutions to the liquid media shortly before use. When producing solid media, the addition was delayed until the solution was hand-hot after autoclaving. The antibiotics listed in Tab. 2.6 were used.

TABLE 2.6
Antibiotics used and concentrations employed

| antibiotic | final concentration | dissolved in |
|---|---|---|
| ampicillin | 100 μg/ml | water |
| chloramphenicol | 20 μg/ml | ethanol |
| gentamicin | 100 μg/ml | ready-for-use (Sigma) |
| kanamycin | 30 μg/ml | water |

2.3.2 Buffer Solutions

| | |
|---|---|
| L-BUFFER: | 50 mM Tris base, 10 mM EDTA, pH 6.8, autoclaving |
| TE: | 10 mM Tris base, 1 mM EDTA, pH 7.4, autoclaving |
| TAE: | 40 mM Tris acetate, 1 mM EDTA, pH 8.0, autoclaving |
| TBE: | 89 mM Tris base, 89 mM boric acid, 2 mM EDTA, |
| | pH 8.0 |
| TBS: | 50 mM Tris base, 137 mM NaCl, 3 mM KCl, pH 7.4, |
| | autoclaving |
| TBS-TWEEN: | TBS + 0.05% Tween-20 |
| PBS: | 137 mM NaCl, 3 mM KCl, 8 mM Na₂HPO₄, 2 mM |
| | KH₂PO₄, pH 7.0, autoclaving |

2.4 Alanine Dehydrogenase Assays

2.4.1 Qualitative Assay

Qualitative detection of AlaDH is based on a number of redox reactions in accordance with the following reaction scheme (Inagaki et al., 1986; Andersen et al., 1992):

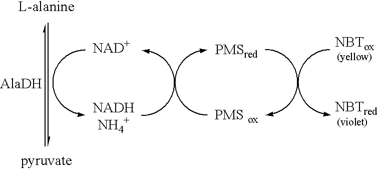

Principle of the Alanine Dehydrogenase Assay

The violet end product can be seen very well with the naked eye in this case. This assay was used, on the one hand, for rapid screening of FPLC fractions and, on the other hand, to demonstrate AlaDH activity in native protein gels.

The basis of this assay is a reaction mix consisting of 1/2 vol. of 0.5 M glycine. KOH, pH 10.2, and 1/8 vol. each of 0.5 M L-alanine, 6.25 mM NAD⁺, 2.4 mM NBT and 0.64 mM PMS.

For the analysis of protein fractions the substrate mix was added 1:1 to the solution to be tested. Native gels were incubated directly in 10 ml of substrate mix after electrophoresis.

A positive reaction can be seen after 5 minutes at the latest.

2.4.2 Semiquantitative Assay

This assay was used to investigate AlaDH activities in mycobacteria.

The mycobacteria were grown on Löwenstein medium. Bacteria were taken from the slant agar tubes using an inoculating loop, resuspended in water and adjusted to a turbidity equivalent to a McFarland Standard No. 5. For separation of cell aggregates the suspensions were treated in an ultrasound bath for 10 minutes.

Reaction mix (see 2.4.1) was then added 1:1 to the cells and incubation was carried out at RT for 10 minutes. After centrifuging at 20,000 g for 2 minutes, the absorption of the supernatant was measured against the blank value.

A batch to which no L-alanine was added was used as the reference measurement. An absorption change of one unit per minute in this test corresponds approximately to an absorption change of three units per minute in the case of the quantitative assay (measurement at 340 nm, see 2.4.3).

2.4.3 Quantitative Assay

In this assay, the quantitative change in the NADH content was measured directly at 340 nm.

The standard reaction batches had a volume of 1 ml. The composition is shown in Tab, 2.7. The absorption was followed over a period of 10 minutes at 37° C. and 340 nm. The extinction coefficient ε of NADH at 340 nm is 6.22×10⁶ cm²/mol.

The standard batches were varied as stated in the text in order to determine the biochemical properties of the enzyme. Every measured value shown represents the average value of at least two, but normally three, independent measurements.

An AlaDH unit is defined as the amount of enzyme that catalyses in one minute the formation of 1 μmol of NADH in the oxidative dgeamination reaction.

TABLE 2.7
Composition of the quantitative AlaDH assay
The composition of the reaction batch for the oxidative
deamination is shown on the left and that for the reductive
amination is shown on the right.

| oxidative deamination | reductive amination |
|---|---|
| 125 mM glycine.KOH, pH 10.2 | 1 M NH₄Cl/NH₄OH, pH 7.4 |
| 100 mM L-alanine | 20 mM pyruvate |
| 1.25 mM NAD⁺ | 0.5 mM NADH |

3. The Distribution of Alanine Dehydrogenase within the Mycobacteria

Both at the gene level and at the protein level, the next aspect to be investigated was in which mycobacteria an alanine dehydrogenase is present. Based on the virulence, the question here was whether the AlaDH activity correlates with that property.

3.1 In vivo AlaDH Activity

Since AlaDH activity is the exception rather than the rule in the microbe world it was interesting to query whether that enzyme is ubiquitous within the mycobacteria or whether it is restricted to certain species and strains. Thereby, inferences can then be made in turn about questions such as:

Do AlaDH-producing strains have common features in their mode of life?

Does a specific method or phase of growth induce AlaDH production?

How does regulation of the AlaDH occur?

Can other metabolic routes replace the reaction catalysed by AlaDH?

What phenotype would AlaDH mutants have to exhibit?

All available strains were therefore investigated for production of AlaDH activity. The repertoire comprised a total of 44 mycobacterial strains, representing 29 different species. In addition, the two strains Nocardia asteroides and Rhodococcus equi which are closely related to the mycobacteria were tested.

In order for the activities measured in the test system to be compared with one another, all the bacterial suspensions were adjusted to a density corresponding to the turbidity of a McFarland Standard No. 5. At the time of measurement, the strains were in the late exponential phase.

In addition to the AlaDH measurement, a measurement was also carried out in which L-alanine was missing from the reaction batch. The activity of that batch is a measure of other NAD⁺-reducing processes proceeding in parallel. The difference between that batch and the standard batch corresponds to the net AlaDH activity (ΔA₅₉₅ value).

According to the activities measured the strains investigated can be divided into three groups. The first group is that of the strongly positive strains (Tab. 3.1). Combined into that group are the strains that have an AlaDH activity of more than 0.5 ΔA₅₉₅ units in the test system used.

Tab. 3.1: Strains having a Strongly Positive AlaDH Activity

The way in which this assay was carried out is described in 2.4.2.

| strain | AlaDH activity [ΔA₅₉₅] |
|---|---|
| M. marinum 3 | 2.327 |
| M. chelonae 7 | 1.842 |
| M. microti 1 | 0.919 |
| M. tuberculosis H37Rᵥ | 0.592 |

Classified as strongly positive were the two strains that are pathogenic for fish, M. chelonae and M. marinum, and the two likewise pathogenic strains, M. microti and M. tuberculosis H37Rᵥ, the latter being a virulent tuberculosis reference strain.

The second group, that of the moderately positive strains, comprises those having an activity between 0.1 and 0.5 ΔA₅₉₅ units (Tab. 3.2).

Tab. 3.2: Strains having a Moderately Positive AlaDH Activity

The way in which this assay was carried out is described in 2.4.2.

TABLE 3.2
Strains having a moderately positive AlaDH activity
The way in which this assay was carried out is described in
2.4.2

| | AlaDH | | |
|---|---|---|---|
| | activity | | AlaDH activity |
| strain | [ΔA₅₉₅] | strain | [ΔA₅₉₅] |
| M. smegmatis 3 | 0.375 | M. tuberculosis 49 | 0.138 |
| M. ulcerus 1 | 0.369 | M. tuberculosis 130 | 0.118 |
| M. africanum 1 | 0.287 | M. smegmatis 1 | 0.116 |
| M. tuberculosis 118 | 0.210 | M. tuberculosis 132 | 0.111 |
| M. tuberculosis 145 | 0.190 | M. tuberculosis 146 | 0.111 |
| M. intracellulare 1 | 0.155 | M. tuberculosis 1 | 0.110 |

In this group, apart from M. smegmatis, only pathogenic, clinical isolates of M. tuberculosis and other mycobacteria are to be found. Both strains of M. smegmatis tested, however, also exhibit very high NAD⁺-reducing activities in the absence of L-alanine. It is also important to mention at this point that the strain M. smegmatis 1-2c (a derivative of M. smegmatis mc²6; Zhang et al., 1991; Garbe et al., 1994; of Dr. Peadar Ó Gaora, St. Mary's Hospital, London), a strain for genetic studies in mycobacteria, does not exhibit any AlaDH activity, but likewise has a high background activity.

Finally, in the last group, there are listed all the strains found to be negative for AlaDH activity, that is to say that have an activity of less than 0.1 ΔA₅₉₅ units (Tab. 3.3).

TABLE 3.3
Strains without AlaDH activity
The way in which this assay was carried out is described in
2.4.2.

| | AlaDH | | |
|---|---|---|---|
| | activity | | AlaDH activity |
| strain | [ΔA₅₉₅] | strain | [ΔA₅₉₅] |
| N. asteroides 1 | 0.048 | M. bovis BCG 4 | 0.001 |
| M. flavescens 1 | 0.042 | M. terrae 2 | 0.001 |
| M. tuberculosis H37Rₐ | 0.032 | M. tuberculosis 60 | 0 |
| M. nonchromogenium 1 | 0.026 | M. tuberculosis 163 | 0 |
| M. fortuitum 11 | 0.022 | M. gastri 1 | 0 |
| M. asiaticum 1 | 0.021 | M. gordonae 3 | 0 |
| M. bovis BCG 2 | 0.013 | M. kansasii 1 | 0 |
| M. lufu 1 | 0.013 | M. parafortuitum 1 | 0 |
| R. equi 1 | 0.011 | M. peregrinum 1 | 0 |
| M. bovis 3 | 0.010 | M. phlei 1 | 0 |
| M. scrofulaceum 1 | 0.009 | M. phlei 4 | 0 |
| M. intracellulare 5 | 0.007 | M. scrofulaceum 8 | 0 |
| M. thermoresistibile 1 | 0.006 | M. simiae 1 | 0 |
| M. avium 1 | 0.002 | M. vaccae 3 | 0 |
| M. triviale 1 | 0.002 | M. xenopi 7 | 0 |

This by far the largest group mainly comprises opportunistic and non-pathogenic strains, and also the two strains related to the mycobacteria, Nocardia asteroides and Rhodococcus equi. Exceptions were two clinical tuberculosis isolates and the pathogen of bovine Tb, M. bovis, but also the two vaccination strains of M. bovis BCG studied.

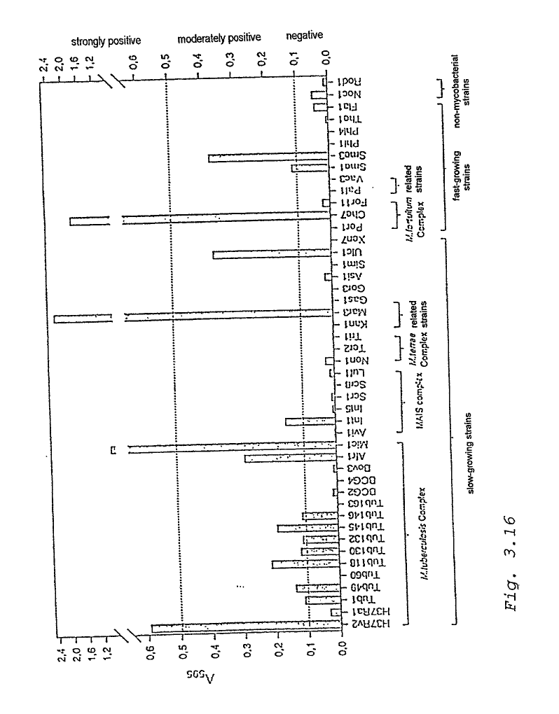

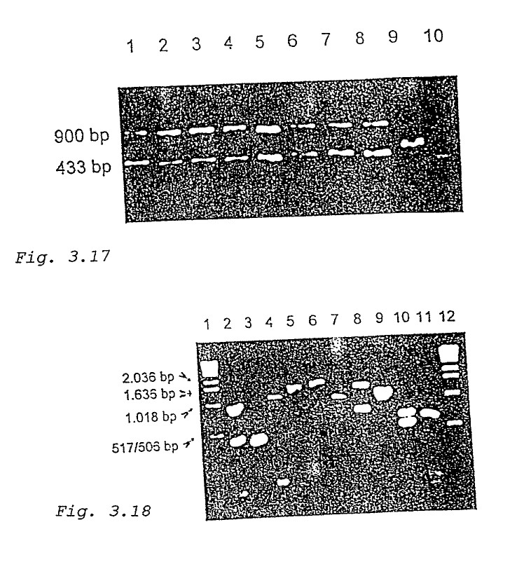

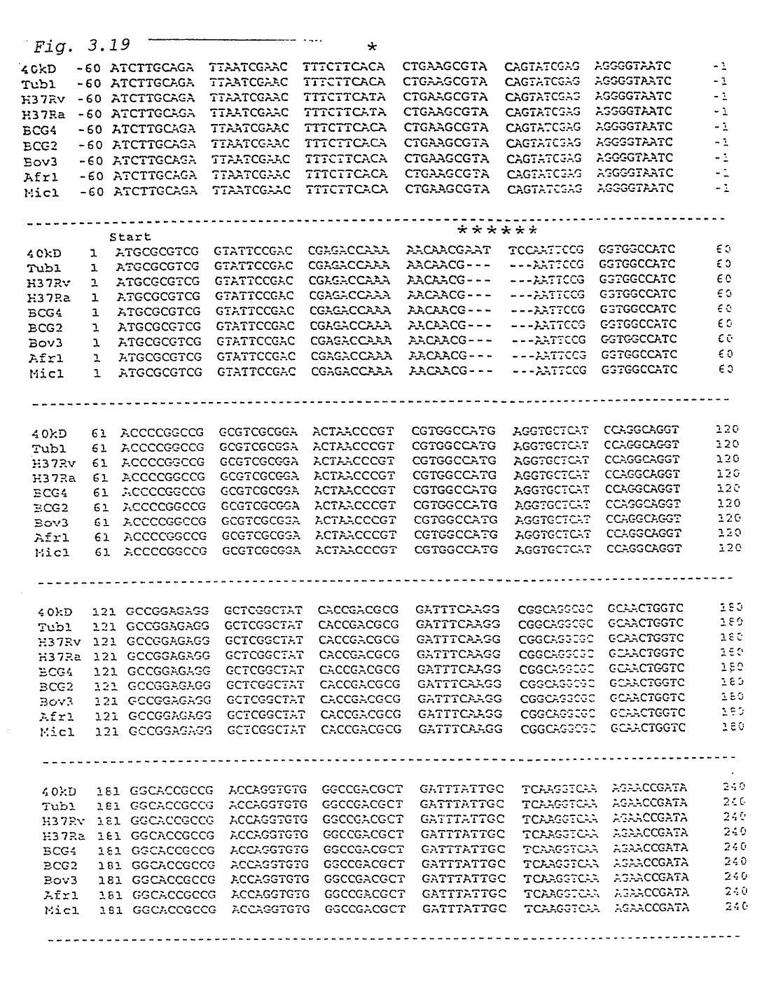

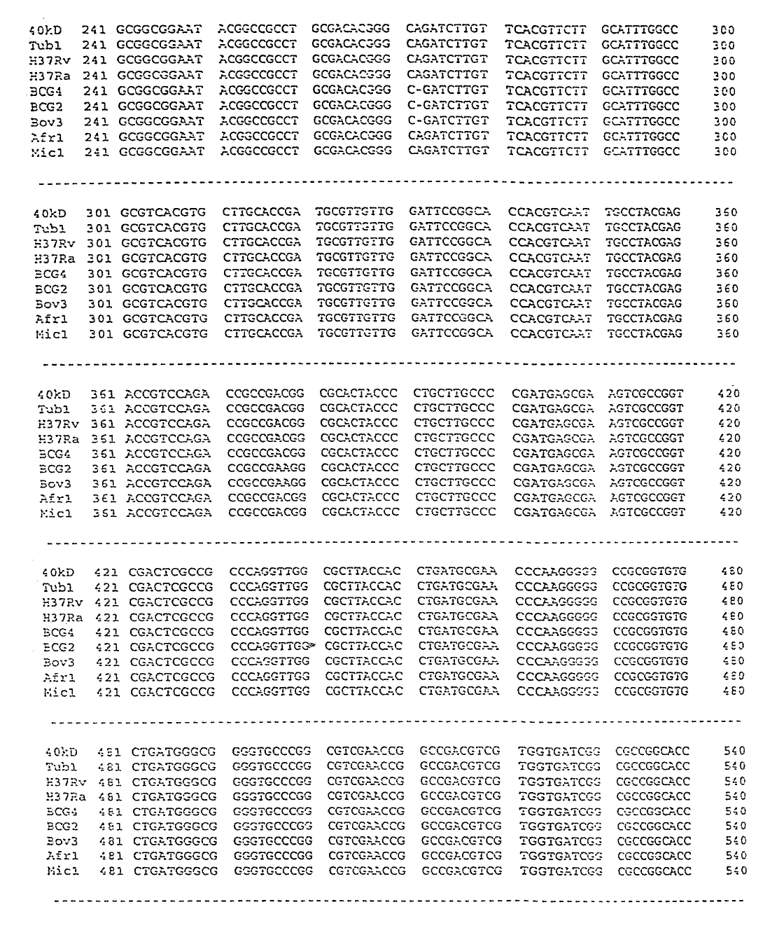

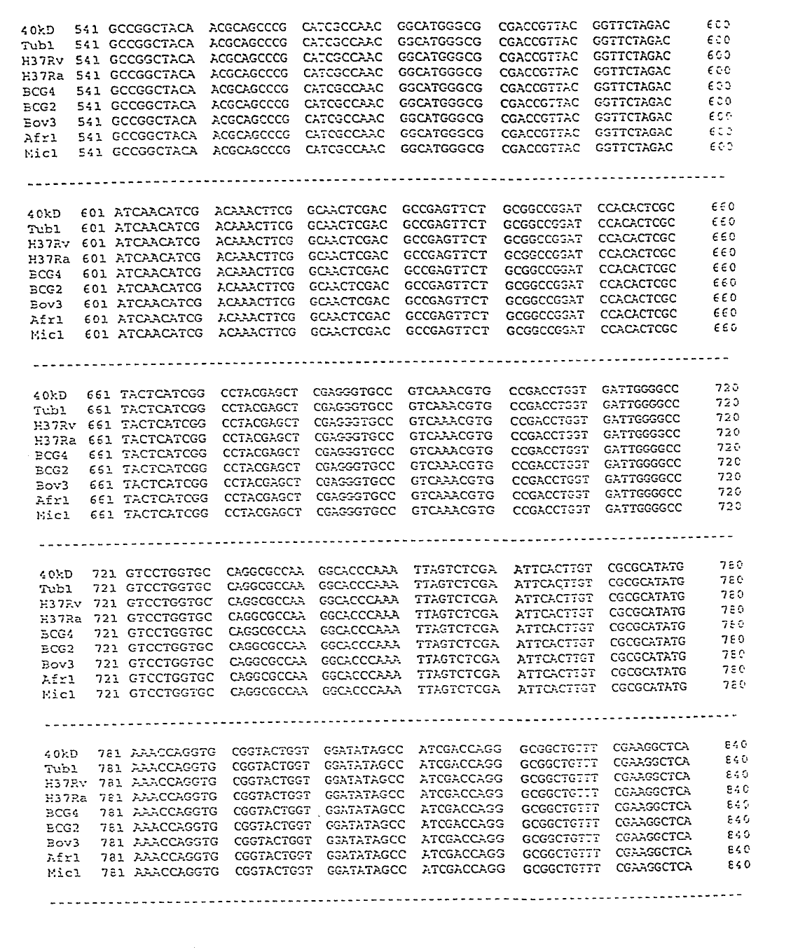

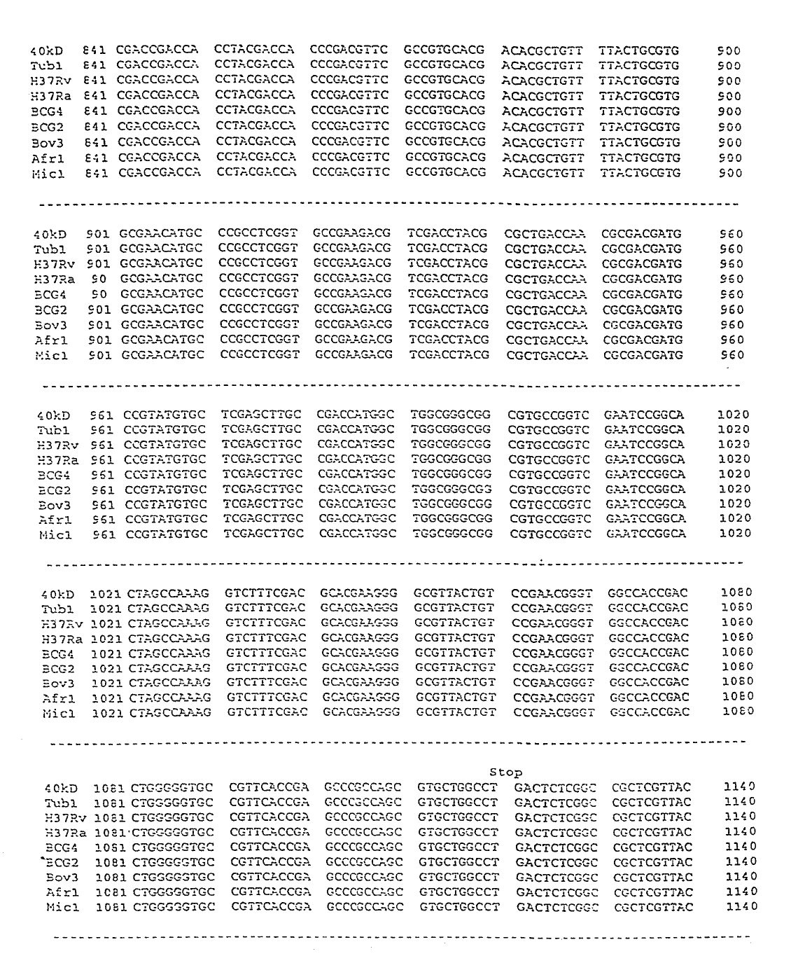

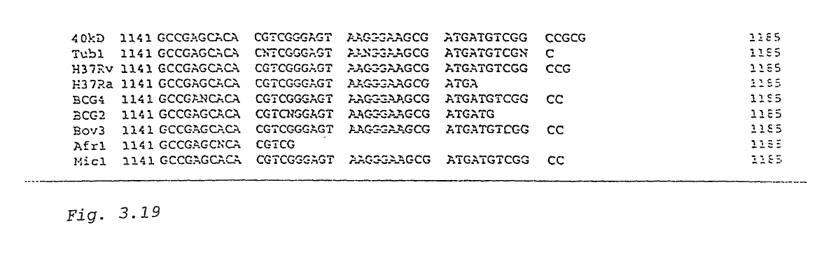

A graph of AlaDH activities in the realm of the mycobacteria is given in FIG. 3.16, ordered according to phylogenetic aspects.

The exact name of the individual strains is given in Tab. 2.3. The statements fast-growing and slow-growing should not be interpreted strictly but, rather, represent a tendency within the groups shown.

To summarise, the distribution of AlaDH activity within the world of the mycobacteria may be described as follows:

1 By far the highest activity is exhibited by the two strains that are pathogenic for fish, M. chelonae and M. marinum.

2 Within the strains of M. tuberculosis there is a tendency that, as virulence decreases, AlaDH activity also decreases (H37Rᵥ>clinical isolates>H37Rₐ)

3 All strains classified as positive are virulent. The only exception is M. smegmatis which, however, is very easily distinguishable on the basis of its high background activity.

4 Not all virulent strains are AlaDH-positive.

5 M. tuberculosis can be distinguished by means of AlaDH activity from the vaccination strain M. bovis BCG.

3.2 The Gene for Alanine Dehydrogenase

3.2.1 The First PCR Fragments

Having quantified the AlaDH activities within the various strains, the next question was why some strains produce the enzyme but others do not. The degree of expression also differs clearly in some cases, even between closely related types.

The absence of measurable activity can to a certain extent be explained by the fact that not all the strains were in exactly the same phase of growth, since it is very difficult to grow all strains parallel, at the same stage. A reason for the absence of activity might, however, also be that genetic changes have an effect on the expression of the gene. Those changes might have occurred in the coding or in the regulatory region.

In order to verify that fact, an attempt was made to amplify the AlaDH gene from various strains, completely or partially, by means of PCR. The primers used for this were oligonucleotides based on the sequence of M. tuberculosis H37Rᵥ (Andersen et al., 1992; see Section 2.2.2, (Tab. 2.5)).

The primer pairs used to detect the AlaDH, the expected length of the respective products and the annealing temperatures of the PCR respectively used are summarised in Tab. 3.4.

TABLE 3.4
Primer pairs for the detection of AlaDH in myco-
bacteria.
The sequences of the primers are given in Tab. 2.5.

| name | primer #1 | primer #2 | product | | temperature |
|---|---|---|---|---|---|
| Annabel | AlaDH-F1 | AlaDH-RM | 433 | bp | 65° C. |
| Beatrice | AlaDH-F1 | AlaDH-R2 | 1102 | bp | 45° C. |
| Claudette | AlaDH-F1 | AlaDH-R3 | 1120 | bp | 55° C. |
| Désirée | AlaDH-F1 | AlaDH-R6 | 1072 | bp | 45° C. |
| Eleonore | AlaDH-F1+ | AlaDH-R1 | 1099 | bp | 55° C. |
| Francoise | AlaDH-F1+ | AlaDH-R2 | 1117 | bp | 50° C. |
| Giselle | AlaDH-F2 | AlaDH-R7 | 757 | bp | 35° C. |
| Helen | AlaDH-F4 | AlaDH-RM | 1080 | bp | 55° C. |
| Isabelle | AlaDH-F4 | AlaDH-R6 | 1050 | bp | 55° C. |
| Jeanette | AlaDH-F5 | AlaDH-R1 | 507 | bp | 45° C. |
| Karen | AlaDH-F5 | AlaDH-R4 | 834 | bp | 45° C. |
| Larissa | AlaDH-F6 | AlaDH-R4 | 786 | bp | 55° C. |
| Melanie | AlaDH-F6 | AlaDH-R5 | 405 | bp | 55° C. |

The first attempts to detect the gene for AlaDH in various mycobacterial species were made with the primer pair Annabel. The result obtained in this case was somewhat surprising. All of the strains of the M. tuberculosis complex exhibited the expected 433 bp fragment. In addition, in all of these strains, an additional fragment of approximately 900 bp had been amplified (FIG. 3.17).

PCR of various strains using the primer pair Annabel.

In these PCRs, 40 cycles having the following sequence were used in each case: melting 2 min at 96° C., annealing 2 min at 65° C. and extension 3 min at 72° C. The MgCl₂ concentration was 1.5 mM.

| | |
|---|---|
| track 1: | M. tuberculosis H37Rᵥ |
| track 2: | M. tuberculosis H37Rₐ |
| track 3: | M. tuberculosis 1 |
| track 4: | M. bovis 3 |
| track 5: | M. bovis BCG 2 |
| track 6: | M. bovis BCG 4 |
| track 7: | M. africanum |
| track 8: | M. microti 1 |
| track 9: | M. marinum 3 |
| track 10: | M. chelonae 7 |

As was to become apparent, that second fragment was also a part of the AlaDH gene, which had come into being as a result of the binding of the primer AlaDH-RM to a site located closer to the C-terminus. By increasing the annealing temperature in the PCR from 65 to 69° C. it was possible to suppress that second fragment (see FIG. 3.18, tracks 2 and 3).

What was actually astounding, however, was the appearance of the amplified fragment in all the strains of the M. tuberculosis complex, irrespective of the existence of AlaDH activity.

In the case of a number of other strains also, it was possible to amplify one or more fragments using the primer pair Annabel. The amplified bands were not, however, particularly strong in most cases and, in view of the 40 PCR cycles, they may therefore be regarded as background. Presumably, weak unspecific reactions are involved. However, the possibility that the PCR primers were unable to bind optimally to the target sequence owing to insufficient homology between the various species also cannot be excluded.

The two fish pathogen strains having a strong AlaDH activity, M. marinum and M. chelonae, exhibited distinctly different behaviours in the PCR with the primer pair Annabel. Whereas M. marinum yielded a product of approximately 540 bp, no fragment could be obtained in the case of M. chelonae under the chosen conditions with the primer pair Annabel (FIG. 3.17, tracks 9 and 10).

3.2.2 The AlaDH Gene of the M. tuberculosis Complex

Since the presence of the gene for AlaDH had been detected in all the strains of the M. tuberculosis complex, the question was how to explain the discrepancy with the measured activities.

For that reason, amplification of larger fragments of the gene was begun. Of M. tuberculosis H37Rᵥ all the fragments listed in Tab. 3.14 could be amplified (some of those fragments are shown in FIG. 3.18). Of the other strains of the M. tuberculosis complex all the PCR reactions from Tab. 3.15 that were tested likewise proceeded positively. Every reaction was not, however, replicated with every strain.

PCR Products of the Strain M. tuberculosis H37Rᵥ

In these PCRs, 40 cycles were used in each case as shown in FIG. 3.17. With the exception of tracks 2 and 3, the annealing temperatures are given in Tab. 3.14. The MgCl₂ concentration in the case of the primer pair Annabel was 1.5 mM, and that in all the other reactions was 3 mM.

| | |
|---|---|
| track 1: | KBL |
| track 2: | Annabel, 65° C. |
| track 3: | Annabel, 69° C. |
| track 4: | Désirée |
| track 5: | Eleonore |
| track 6: | Francoise |
| track 7: | Giselle |
| track 8: | Helen |
| track 9: | Isabelle |
| track 10: | Larissa |
| track 11: | Melanie |
| track 13: | KBL |

The amplified region of all the strains of the M. tuberculosis complex comprises 1260 bp. It contains the complete coding segment for the AlaDH, and a further 75 bp upstream and 63 bp downstream. This region of all the strains of the M. tuberculosis complex was sequenced completely (FIG. 3.19). Only in the last 20 bases or so did inaccuracies creep in. The complete remaining region has, however, been confirmed by repeated sequencing.

It can be ascertained that all the sequences are identical to the published sequence of the λAA65 clone (Andersen et al., 1992) apart from three sites.

Alignment of the AlaDH gene and the flanking regions of various strains of the M. tuberculosis complex

The line designated “40 kD” gives the sequence of Andersen et al. (1992). Sequence differences are each marked with a “*” above the sequence. The start and stop codons are also marked above the sequence. The bases printed in bold typeface at the end of the sequence are sequencing inaccuracies.

The first site at which the sequences differ is base −32, that is to say upstream of the translation start signal. Interestingly, the sequences of M. tuberculosis H37Rᵥ and H37Rₐ determined in this study differ from the sequence of Andersen and co-workers (Andersen et al., 1992) at that site. All the other sequences investigated in this study, including that of the third strain of M. tuberculosis tested, agree with the sequence of Andersen.

This is astonishing, given that the originally published sequence is based on the clone of a λgt11 bank that had been produced from the strain M. tuberculosis H37Rᵥ. The question of whether an error had perhaps been introduced by the PCR was therefore investigated. That, however, did not prove to be correct. It might also be possible, however, that the strain of M. tuberculosis H37Rᵥ used in this study had a different origin from that of Andersen. Similar small variations are also known in the case of various M. bovis BCG strains of different origins.

At the second site, all strains of the M. tuberculosis complex differ from the published sequence of the AlaDH of M. tuberculosis H37Rᵥ. The region concerned is that of bases 38 to 49. Within those twelve bases the sequence AATTCC is repeated; bases 44 to 49, therefore, represent a direct repeat of bases 38 to 43. In all eight of the strains sequenced, that pattern is to be found, however, only once in each. It is therefore to be assumed that a sequencing or reading error has crept in in the case of the sequence determined by Andersen et al. (1992). As a result, the gene sequence and the amino acid sequence derived therefrom changes as follows: Andersen et al., 1992:

```
gene sequence
A A C G A A T T C C A A T T C C G G G T G
protein sequence
  Asn Glu Phe Gln Phe Arg Val
This study:
gene sequence
A A C G A A T T C - - - - - - C G G G T G
protein sequence
  Asn Glu Phe - - Arg Val
```

What is effectively involved, therefore, is the “loss” of the two amino acids glutamine and phenylalanine. After that deletion, the sequence continues as published by Andersen et al. (1992).

That fact was confirmed by N-terminal sequencing of the protein. Neither in the native protein of M. tuberculosis H37Rᵥ nor in the recombinant protein from E. coli were the two amino acids to be found.

The third site that differs is base 272. At that site, with the exception of three strains, there is an adenine residue. In the case of those three strains, M. bovis and two strains of M. bovis BCG, that base has been deleted. The deletion leads to a reading frame shift that affects the entire following part of the resulting protein. As a result of that reading frame shift, an opa1 stop signal occurs at bases 404 to 406. The product of that gene is therefore only about one third the size of the functional AlaDH of the other strains.

What is decisive in the case of this third discrepancy in the gene sequence is the fact that it occurs in precisely the three strains that do not exhibit any AlaDH activity. M. bovis and M. bovis BCG are the only strains of the M. tuberculosis complex that do not exhibit any activity. All the other strains were classified as being moderately or strongly positive. The observed deletion, therefore, is the reason for the absence of a functional AlaDH. Since, however, the truncated protein also could not be detected with the mAb HBT-10 (the epitope of HBT-10 lies in the region before the reading frame shift), it is to be assumed that the truncated protein is not produced in the first place or is produced only in very small amounts that are not detectable with the mAb HBT-10.

4 AlaDH Activity and AlaDH Gene in Mycobacteria

AlaDH activity in mycobacteria. The AlaDH activities measured permit a number of interesting observations regarding the mode of life of the organisms that have a positive activity.

The strains that have a strong activity are all pathogenic. It is interesting here that two of the four strains falling into that group are pathogenic for fish (Austin & Austin, 1987). Both of those, M. marinum and M. chelonae, can, however, infect humans also (Wallace et al., 1983; Johnston & Izumi, 1987). In contrast to tuberculosis, however, they cause morbid infections of the upper layers of the skin in most cases, which are relatively unproblematical to treat in most cases.

M. chelonae is a comparatively fast-growing, non-chromogenic bacterium. Infections in humans often occur in the form of secondary wound infections following operations (Cooper et al., 1989). M. marinum is a slow-growing organism that forms a yellow pigment when growing in light. Infections with M. marinum have been detected in more than 50 poikilothermic species (reptiles, amphibians, fish). In humans, the bacterium usually manifests itself in the elbow or knee area.

The two other strains having a strongly positive AlaDH activity are representatives of the M. tuberculosis complex. They are the tuberculosis reference strain, M. tuberculosis H37Rᵥ, and the strain M. microti, which is regarded as a phylogenetic link between M. tuberculosis and M. bovis.

With the exception of M. smegmatis, all of the strains classified as moderately positive also are pathogenic. The majority of those strains comprises clinical isolates of M. tuberculosis. Pathogenic variants of tuberculosis strains appear, therefore, to have AlaDH activity as a rule. Two isolates were also found, however, that did not exhibit any AlaDH activity. The only non-pathogenic organism having AlaDH activity is the fast-growing strain M. smegmatis. M. smegmatis is characterised, however, by an unusually high NAD⁺-reducing background activity and is therefore very easily distinguished from all the other strains having AlaDH activity. Furthermore, in the strain M. smegmatis 1-2c, a mycobacterial expression strain, no AlaDH activity was found.

Within the 44 mycobacteria strains tested, and that is by far the majority of all known strains, the following conclusion is therefore permissible:

a slow-growing mycobacterium having positive AlaDH activity is virulent.

The converse of that statement is, however, false. Among the strains that do not have AlaDH activity, several are virulent. Nevertheless, one cannot help finding a tendency, although not strong, for AlaDH activity to increase with increasing pathogenicity of a strain. That thesis is lent greater weight especially by the activities of the various strains of M. tuberculosis. By far the highest activity is exhibited by the strain H37Rᵥ, which serves as the reference strain for all tuberculosis laboratories and which is known to be highly infectious. At the very end of the scale there is the avirulent derivative of H37Rᵥ, the strain H37Rₐ. Ranged between those two poles are the clinical tuberculosis isolates, some of which exhibit slightly more activity and some slightly less.

The AlaDH gene in mycobacteria. The gene for alanine dehydrogenase could be identified in all the strains of the M. tuberculosis complex investigated and in the strain M. marinum.

The decisive point when comparing the sequences within the M. tuberculosis complex is the deletion of base 272 which, in the case of the strains of M. bovis and M. bovis BCG investigated, result in a reading frame shift and ultimately in a truncated, non-functional protein. In the case of those strains, no AlaDH activity could be detected in cell extracts either. Those data also agree with the results of Andersen et al. (1992) who obtained signals with those strains in Southern blots but could not detect any protein in Western blots.

By amplifying and sequencing the gene it was possible in this study to find the reason for this. It is also necessary to take into consideration, however, that other changes in the regulatory gene segments may be responsible for the absence of the truncated protein. This might be a measure taken by the cell not to invest energy in a protein that is not capable of functioning. In general, not much is known yet about regulatory gene sequences in mycobacteria (Dale & Patki, 1990; Gupta et al., 1993). It appears, however, that, in accordance with the principle of enhancers, segments located further away may also have a not inconsiderable influence on the gene expression. The mutations required for a regulation of the production of the protein do not necessarily have to lie, therefore, in the region sequenced in this study.

The other AlaDH gene identified, that of M. marinum, is clearly different at the DNA level from the genes of the M. tuberculosis complex. Nevertheless, four of five bases (80.4%) are, however, still identical on average upon comparison of those sequences. That value is even higher at the protein level (85.3% identity, 92.0% similarity). Since, however, AlaDH activity has also been found in a number of other species, it is to be assumed that the corresponding genes could not be amplified under the conditions used for lack of homology to the primers used. A more detailed study with regard to that point should be able to find those genes also. A comparison of all those sequences might allow further conclusions to be drawn on the role of the enzyme.

It is furthermore conceivable that, using such a sequence comparison, it should be possible to develop a PCR process with which mycobacteria that have an AlaDH gene can be distinguished from one another. And, as it has been possible to show in this study, it is precisely the strains that are of importance to humans that possess an AlaDH gene. Especially the possibility of being able to distinguish the pathogen M. tuberculosis from the vaccination strain M. bovis BCG using such a PCR assay makes such a project appear interesting.

Prospects. The 40 kD antigen with which this study has been concerned is a worthwhile subject for more detailed investigations in several respects. One aspect that has not been considered in detail in this study is the possible use of that enzyme in medical diagnostics. For example, assays that are based on an AlaDH have already been described for the enzymes dipeptidase (Ito et al., 1984), γ-glutamyltransferase (Kondo et al., 1992) and γ-glutamyl cyclotransferase (Takahashi et al., 1987). All three of the enzymes mentioned are to be found in altered urine, serum and/or blood concentrations in various diseases.

The main attention, however, is on the use of the 40 kD antigen in the case of tuberculosis. Several points from which this can be approached are conceivable.

In diagnostics alone, it is possible to envisage several possible ways in which the 40 kD antigen or its underlying gene might be used. Since the recombinant protein can easily be recovered from the overproducing E. coli strain, it appears worthwhile to study the usefulness of that protein in serology. In addition, it might be possible to develop diagnostic processes based on the direct detection of AlaDH activity or, as already mentioned, on amplification of specific parts of the gene. The deletion of base 272 in the strains M. bovis and M. bovis BCG may serve here as the starting point for discrimination of those two strains from M. tuberculosis.

It also should be possible to create a PCR assay for the strain M. marinum which, of course, at the gene level, differs not inconsiderably from the M. tuberculosis complex. Up to now, a PCR assay relying on amplification of a part of the gene sequence coding for the 16S rRNA has been used for that purpose (Knibb et al., 1993). This is of great importance in view of the increasing number of infections with M. marinum in fish farms in recent years. Infections in humans also have been reported more frequently in recent years (Harris et al., 1991; Kullavanijaya et al., 1993; Slosarek et al., 1994).

The observation that the virulence of a strain of M. tuberculosis correlates very well with its AlaDH activity again poses the question whether the enzyme represents a virulence factor.

To answer that question, approaches such as knock-out of the gene in M. tuberculosis or overexpression of the gene in a strain of low virulence are conceivable. In both cases, the virulence can be tested in an animal model.

The disclosure also includes all conceivable combinations of the individual features disclosed.

6. Appendices

List of Abbreviations

| | |
|---|---|
| A | pre-exponential factor or impact factor |
| Aₓₓₓ | absorption at a wavelength of xxx nm |
| AlaDH | L-alanine dehydrogenase (E.C. 1.4.1.1.) |
| AMC | Academic Medical Centre, Amsterdam, The Netherlands |
| Ap | ampicillin |
| AP | alkaline phosphatase |
| app. | apparent |
| AS | amino acid |
| ATCC | American Type Culture Collection, Rockville, USA |
| ATP | adenosine triphosphate |
| BCG | Bacille Calmette Guérin |
| BCIG | 5-bromo-4-chloro-3-indolyl-β-D-galactopyranoside |
| BCIP | 5-bromo-4-chloro-3-indolyl phosphate |
| Boc | tert-butoxycarbonyl |
| bp | base pair(s) |
| cfu | colony forming units |
| Cm | chloramphenicol |
| Conc | concentration |
| DMEM | Dulbecco's Modified Eagle Medium |
| DMF | dimethylformamide |
| DMSO | dimethyl sulphoxide |
| DNA | deoxyribonucleic acid |
| DTNB | dithiobisnitrobenzoic acid |
| DTT | dithiothreitol |
| Eₐ | activation energy |
| EDTA | ethylenediamine tetraacetate |
| Eth | ethionamide |
| F | farad |
| f.a. | for analysis, of the highest degree of purity |
| FBS | foetal bovine serum |
| FCS | foetal calf serum |
| Fmoc | 9-fluorenylmethoxycarbonyl |
| FPLC | Fast Protein Liquid Chromatrography |
| frag. | fragment |
| g | acceleration due to gravity |
| GBF | Gesellschaft für biotechnologische Forschung mbH, |
| | Braunschweig, Germany |
| GlcNAc | N-acetylglucosamine |
| Gm | gentamicin |
| GOGAT | glutamine oxoglutarate aminotransferase |
| GS | glutamine synthetase |
| GST | glutathione S-transferase |
| h | hour(s) |
| HBSS | Hank's Balanced Salt Solution |
| HIV | Human Immunodeficiency Virus |
| HOBt | hydroxybenzotriazole |
| HRP | horseradish peroxidase |
| Hsp | heat shock proteins |
| Ig | immunoglobulin |
| IL | interleukin |
| INH | isonicotinic acid hydrazide, isoniazide |
| IPTG | isopropyl-β-D-thiogalactoside |
| k | conversion rate of an enzyme |
| kb | kilobases |
| KBL | kilobase ladder |
| kD, kDa | kilodalton |
| KIT | Royal Tropical Institute, Amsterdam, The Netherlands |
| K_{M} | Michaelis constant |
| Km | kanamycin |
| MΦ | macrophage(s) |
| mAb | monoclonal antibody |
| MAIS | M. avium-M. intracellulare-M. scrofulaceum |
| | complex |
| MBP | maltose binding protein |
| MCAC | metal chelate affinity chromatography |
| mesoDAP | meso-diaminopimelic acid |
| min | minute(s) |
| m.o.i. | multiplicity of infection |
| MRC | Medical Research Council, Tuberculosis and Related |
| | Infections Unit, London, England |
| MTT | thiazolylblue tetrazolium bromide |
| MurNAc | N-acetylmuramic acid |
| MurNG1 | N-glycolylmuramic acid |
| NAD⁺ | nicotinamide adenine dinucleotide, oxidised form |
| NADH | nicotinamide adenine dinucleotide, reduced form |
| NADP⁺ | nicotinamide adenine dinucleotide phosphate, oxidised |
| | form |
| NADPH | nicotinamide adenine dinucleotide phosphate, reduced |
| | form |
| n.d. | not determined |
| NBT | nitroblue tetrazolium chloride |
| No. | number |
| NTP | any nucleotide in the form of a triphosphate |
| oD | oxidative deamination |
| ON | overnight |
| ORF | open reading frame |
| OtBu | tert-butyl ester |
| PAGE | polyacrylamide gel electrophoresis |
| pac | protein antigen c, old term for the 40 kD antigen |
| PCR | polymerase chain reaction |
| Pfp | pentafluorophenyl |
| PMA | phorbol myristate acetate |
| Pmc | pentamethylchromane |
| PMS | phenazine methosulphate |
| PNT | pyridine nucleotide transhydrogenase |
| PPD | purified protein derivative |
| PVDF | polyvinylidene difluoride |
| R | Rydberg constant or resistance (when superscript |
| | letter) |
| rA | reductive amination |
| rec | recombinant |
| Rha | rhamnose |
| Rif | rifampicin |
| RIV | National Institute of Public Health and the |
| | Environment, Buthoven, The Netherlands |
| RNA | ribonucleic acid |
| RNI | reactive nitrogen intermediates |
| ROI | reactive oxygen intermediates |
| rpm | revolutions per minute |
| rRNA | ribosomal ribonucleic acid |
| RT | room temperature |
| SDS | sodium dodecyl sulphate |
| sec | second(s) |
| Str | streptomycin |
| Tb | tuberculosis |
| TEMED | N,N,N′,N′-tetramethylethylenediamine |
| TIR | translation initiation region |
| Tris | tris (hydroxymethyl) aminomethane |
| Trt | trityl |
| ts | temperature-sensitive |
| Tween | polyoxyethylenesorbitan monolaurate |
| U | unit(s) |
| Vₘₐₓ | maximum reaction velocity |
| VMDC | Veterinary Microbiological Diagnostic Centre, |
| | Utrecht, The Netherlands |
| vol. | volume |
| WHO | World Health Organisation |
| WKZ | Academisch Ziekenhuis, Utrecht, The Netherlands |

Abbreviations for Amino Acids and Nucleotides

| amino acid | 3-letter code | 1-letter code |
|---|---|---|
| alanine | Ala | A |
| arginine | Arg | R |
| asparagine | Asn | N |
| aspartate | Asp | D |
| cysteine | Cys | C |
| glutamine | Gin | Q |
| glutamate | Glu | E |
| glycine | Gly | G |
| histidine | His | H |
| isoleucine | Ile | I |
| leucine | Leu | L |
| lysine | Lys | K |
| methionine | Met | M |
| phenylalanine | Phe | F |
| proline | Pro | P |
| serine | Ser | S |
| threonine | Thr | T |
| tryptophan | Trp | W |
| tyrosine | Tyr | Y |
| valine | Val | V |
| | nucleoside/ | |
| base | nucleotide | abbreviation |
| adenine | adenosine | A |
| cytosine | cytidine | C |
| guanine | guanosine | G |
| uracil | uridine | U |
| thymine | thymidine | T |

### 6. BIBLIOGRAPHY

Andersen A. B.; Andersen P.& Ljungqvist L. (1992). Structure and Function of a 40,000-Molecular-Weight Protein Antigen from Mycobacterium tuberculosis. Infect. lmmun. 60, 2317-2323.

Austin B. & Austin D. A. (1987). Bacterial Fish Pathogens—Disease in Farmed and Wild Fish, Chapter 7: Aerobic Gram-Positive Rods. Ellis Horwood Ltd., Chicester.

Bass Jr. J. B.; Farer L. S.; Hopewell P. C.; Jacobs R. F. & Snider Jr. D. E. (1990). Diagnostic Standards and Classification of Tuberculosis. Am.Rev.Resp.Dis. 142, 725-735.

Cherayil B. J. & Young R. A. (1988). A 28-kDa Protein from Mycobacterium leprae Is a Target of the Human Antibody Response in Lepromatous Leprosy. J.Immunol. 141, 4370-4375.

Clark H. F. & Shepard C. C. (1963). Effect of Environmental Temperatures on Infection with Mycobacterium marinum (balnei) of Mice and a Number of Poikilothermic Species. J.Bacteriol. 86, 1057-1069.

Cooper J. F.; Lichtenstein M. J.; Graham B. S. & Schaffner W. (1989). Mycobacterium chelonae: A Cause of Nodular Skin Lesions with a Proclivity for Renal Transplant Recipients. Am.J.Med. 86, 173-177.

Dale J. W. & Patki A. (1990). Mycobacterial Gene Expression and Regulation. In: Molecular Biology of the Mycobacteria. (Ed. J. McFadden) Surrey University Press, London.

Delforge D.; Depiereux E.; De Bolle X.; Feytmans E. & Remacle J. (1993). Similarities Between Alanine Dehydrogenase and the N-Terminal Part of Pyridine Nucleotide Transhydrogenase and their Possible Implication in the Virulence Mechanism of Mycobacterium tuberculosis. Biochem.Biophys. Res.Comm. 190, 1073-1079.

van Eden W.; Thole J. E.; van der Zee R.; Noordzij A.; van Embden J. D.; Hensen E. J. & Cohen I. R. (1988). Cloning of the Mycobacterial Epitope Recognized by T lymphocytes in Adjuvant Arthritis. Nature 331, 171-173.

Garbe T. R.; Barathi J.; Barnini S.; Zhang Y.; Abou-Zeid C.; Tang D.; Mukherjee R. & Young D. B. (1994). Transformation of a Range of Mycobacterial Species using Hygromycin as Selectable Marker. Mol.Microbiol. 140, 133-138.

Gottesman M. E.; Adhya S. & Das A. (1980). Transcription Antitermination by Bacteriophage Lambda N Gene Product. J.Mol. Biol. 140, 57-75.

di Guan C.; Li P.; Riggs P. D. & Inouye H. (1987). Vectors that Facilitate the Expression and Purification of Foreign Peptides in Escherichia coli by Fusion to Maltose-Binding Protein. Gene 67, 21-30.

Gupta S. K.; Bashyam M. D. & Tyagi A. K. (1993). Cloning and Assessment of Mycobacterial Promoters by Using a Plasmid Shuttle Vector. J.Bacteriol. 175, 5186-5192.

Gupta S. & Tyagi A. K. (1993). Sequence of a Newly Identified Mycobacterium tuberculosis Gene Encoding a Protein with Sequence Homology to Virulence-Regulating Proteins. Gene 126, 157-158.

Hanahan D. (1983). Studies on Transformation of Escherichia coli with Plasmids. J.Mol.Biol. 166, 557-580.

Harris L. F.; Striplin W. H. & Burnside R. C. (1991). Aquatic Hazard Mycobacterium marinum Infection. Ala.Med. 61, 8-10.

Huebner R. E.; Schein M. F. & Bass Jr. J. B. (1993). The Tuberculin Skin Test. Clin.Inf.Dis. 17, 968-975.

Inagaki K.; Tanizawa K.; Badet B.; Walsh C. T.; Tanaka H. & Soda K. (1986). Thermostable Alanine Racemase from Bacillus stearothermophilus: Molecular Cloning of the Gene, Enzyme Purification and Characterization. Biochemistry 25, 3268-3274.

Ito Y.; Watanabe Y.; Hirano K.; Suguira M.; Sawaki S. & Ogiso T. (1984). A Fluorometric Method for Dipeptidase Activity Measurement in Urine, Using L-Alanyl-L-Alanine as Substrate. J.Biochem. 96, 1-8.

Johnston J. M. & Izumi A. K. (1987). Cutaneous Mycobacterium marinum Infection (“Swimming Pool Granuloma”). Clin.Dermatol. 5, 68-75.

Khanolkar-Young S.; Kolk A. H. J.; Andersen A. B.; Bennedsen J.; Brennan P. J.; Rivoire B.; Kuijper S.; McAdam K. P. W. J.; Abe C.; Batra H. V.; Chaparas S. D.; Damiani G.; Singh M. & Engers H. D. (1992). Results of the Third Immunology of Leprosy/Immunology of Tuberculosis Antimycobacterial Monoclonal Antibody Workshop. Infect. Immun. 60, 3925-7.

Knibb W.; Colorni A.; Ankaoua M.; Lindell D.; Diamant A. & Gordin H. (1993). Detection and Identification of a Pathogenic Marine Mycobacterium from the European Seabass Dicentrarchus labrax Using Polymerase Chain Reaction and Direct Sequencing of 16S rDNA Sequences. Mol.Mar.Biol. Biotechnol. 2, 225-232.

Kondo H.; Hashimoto M.; Nagata K.; Tomita K. & Tsubota H. (1992). Assay of γ-Glutamyltransferase with Amino Acid Dehydrogenases from Baci!lus stearothermophilus as Auxiliary Enzymes. Clin.Chim.Acta 207, 1-9.

Kullavanijaya P.; Sirimachan S. & Bhuddhavudhikrai P. (1993). Mycobacterium marinum Cutaneous Infections Acquired from Occupations and Hobbies. Int.J.Dermatol. 32, 504-507.

McCarthy J. E. G.; Sebald W.; Gross G. & Lammers R. (1986). Enhancement of Translation Efficiency by the Escherichia coli atpE Translation Initiation Region: Its Fusion with Two Human Genes. Gene 41, 201-206.

Mehra V; Sweetser D. & Young R. A. (1986). Efficient Mapping of Protein Antigenic Determinants. Proc.Natl.Acad.Sci. 83, 7013-7017.

Mehra V.; Bloom B. R.; Bajardi A. C.; Grisso C. L.; Sieling P. A.; Alland D.; Convit J.; Fan X. D.; Hunter S. W. & Brennan P. J.; Rea T. H. & Modlin R. L. (1992). A Major T Cell Antigen of Mycobacterium leprae Is a 10-kD Heat-Shock Cognate Protein. J.Exp.Med. 175, 275-284.

Ralph P. & Nakoinz I. (1975). Phagocytosis and Cytolysis by a Macrophage Tumor and its Cloned Cell Line. Nature 257, 393-394.

Ralph P. & Nakoinz I. (1976). Lysozyme Synthesis by a Established Human and Murine Histiocytic Lymphoma Cell Line. J.Exp. Med. 143, 1528-1533.

Sambrook J.; Fritsch E. F. & Maniatis T. (1989). Molecular Cloning—A Laboratory Manual, Second Edition. Cold Spring Harbor Laboratory Press, Cold Spring Harbor.

Schauder B.; Blöcker H.; Frank R. & McCarthy J. E. G. (1987). Inducible Expression Vectors Incorporating the Escherichia coli atpE Translation Initiation Region. Gene 52, 279-283.

Singh M.; Andersen A. B.; McCarthy J. E. G.; Rohde M.; Schotte H.; Sanders E. & Timmis K. N. (1992). The Mycobacterium tuberculosis 38-kDa antigen: Overproduction in Escherichia coli, Purification and Characterization. Gene 117, 53-60.

Slosarek M.; Kubin M. & Pokorny J. (1994). Water as a Possible Factor of Transmission in Mycobacterial Infections. Cent.Eur.J.Public Health 2, 103-105.

Snyderman R.; Pike M. C.; Fischer D. G. & Koren H. S. (1977). Biologic and Biochemical Activities of Continuous Macrophage Cell Lines P338D1 and J774.1. J.Immunol. 119, 2060-2066.

Studier F. W. (1975). Genetic Mapping of a Mutation that Causes Ribonuclease III Deficiency in Escherichia coli. J.Bacteriol. 124, 307-316.

Takahashi T.; Kondo T.; Ohno H.; Minato S.; Ohshima T.; Mikuni S.; Soda K. & Taniguchi N. (1987). A Spectrophotometric Method for the Determination of γ-Glutamyl Cyclotransferase with Alanine Dehydrogenase in the Presence of Anthglutin. Biochem.Med.Metabol.Biol. 38, 311-316.

Thole J. E.; Keulen W. J.; de Bruyn J.; Kolk A. H.; Groothuis D. G.; Berwald L. G.; Tiesjema R.H. & van Embden J. D. (1987). Characterization, Sequence Determination, and Immunogenicity of a 64-Kilodalton Protein of Mycobacterium bovis BCG Expressed in Escherichia coli K-12. Infect.Immun. 55, 1466-1475.

Thole J. E.; Stabel L. F.; Suykerbuyk M. E.; de Wit M. Y.; Klatser P. R.; Kolk A. H. & Hartskeerl R. (1990). A Major Immunogenic 36,000-Molecular-Weight Antigen from Mycobacterium leprae Contains an lmmunoreactive Region of Proline-Rich Repeats. Infect.Immun. 58, 80-87.

Verbon A.; Hartskeerl R. A.; Schuitema A.; Kolk A. H. J.; Young D. B. & Lathigra R. (1992). The 14,000-Molecular-Weight Antigen of Mycobacterium tuberculosis Is Related to the AlphaCrystallin Family of Low-Molecular-Weight Heat Shock Proteins. J.Bacteriol. 174, 1352-1359.

Vordermeier H. M.; Harris D. P.; Lathigra R.; Roman E.; Moreno C. & Ivanyi J. (1993). Recognition of Peptide Epitopes of the 16,000 MW Antigen of Mycobacterium tuberculosis by Murine T Cells. Immunology 80, 6-12.

Wallace Jr. R. J.; Swenson J. M.; Silcox V. A.; Good R. C.; Tschen J. A. & Stone M. S. (1983). Spectrum of Disease Due to Rapidly Growing Mycobacteria. Rev.Infect.Dis. 5, 657-679.

Zhang Y.; Lathigra R.; Garbe T.; Catty D. & Young D. (1991). Genetic Analysis of Superoxide Dismutase, the 23 Kilodalton Antigen of Mycobacterium tuberculosis. Mol.Microbiol. 5, 381-391.

1. An enzymatic test kit for the diagnosis of tuberculosis and other mycobacterial infections in humans and animals by determination of the activity of alanine dehydrogenase (E.C. 1.4.1.1), comprising L-alanine, nicotinamide adenine dinucleotide (oxidised form; NAD⁺), phenazine methosulphate (PMS) and nitroblue tetrazolium chloride (NBT).

2. A method for the diagnosis of tuberculosis and other mycobacterial infections of humans and animals, characterised in that the activity of alanine dehydrogenase (E.C. 1.4.1.1.) is measured with an enzymatic test kit according to claim 1.

3. A method according to claim 2, characterised in that
(i) possible tuberculosis pathogens, such as M. tuberculosis, are isolated,
(ii) a crude cell extract is made,
(iii the extract is incubated in solution and
(iv) the absorption is measured.

4. A method according to claim 2 and/or 3, characterised in that clinical samples, such as body fluids, are subjected directly to tuberculosis diagnosis and the alanine dehydrogenase activity is measured.

5. A method according to claim 2, characterised in that cells, strains and/or species of disease-causing organisms (mycobacteria) are differentiated from non-virulent cells and strains.

6. A method according to claim 5, characterised in that cells, strains and/or species of disease-causing organisms of the M. tuberculosis complex are identified and differentiated.

7. A method according to any one of the preceding claims, characterised in that the method is carried out in the presence of substances that inhibit tuberculosis and other mycobacterial infections of humans and animals and those inhibiting substances are optionally recovered.

8. A method according to any one of the preceding claims, characterised in that it is carried out
(i) to control epidemics and/or
(ii) after vaccinations (vaccination follow-up) in humans and animals.

9. A DNA sequence selected from the following group or other partial sequences of the alanine dehydrogenase gene of M. tuberculosis (FIG. 2.5):

| | | Orienta- |
|---|---|---|
| Name | Sequence | tion |
| AlaDH-F1 | 5′-ATGCGCGTCGGTATTCCG-3′ | forward |
| AlaDH-F1+ | 5′-GCGCGTCGGTATTCCGACCG-3′ | forward |
| AlaDH-F2 | 5′-GAGACCAAAACAACGAA-3′ | forward |
| AlaDH-F4 | 5′-GAATTCCCATCAGCAATCTTGCAGA-3′ | forward |
| AlaDH-F5 | 5′-GCCCCGATGAGCGAAGTC-3′ | forward |
| AlaDH-F6 | 5′-GGGGCCGTCCTGGTGCC-3′ | forward |
| AlaDH-F7 | 5′-GACGTCGACCTACGCGCTGAC-3′ | forward |
| AlaDH-R1 | 5′-CTCGGTGAACGGCACCCC-3′ | reverse |
| AlaDH-R2 | 5′-GGCCAGCACGCTGGCGGG-3′ | reverse |
| AlaDH-R3 | 5′-CACCCGTTCGGACAGTAA-3′ | reverse |
| AlaDH-R4 | 5′-CGCGGCCGACATCATCGC-3′ | reverse |
| AlaDH-R5 | 5′-GGCCGACATCATCGCTTCCC-3′ | reverse |
| AlaDH-R6 | 5′-CGAGACTAATTTGGGTGCCTTGGC-3′ | reverse |
| AlaDH-R7 | 5′-ATTTGGGTGCCTTGGC-3′ | reverse |
| AlaDH-RM | 5′-GGCGGCGAGTCGACCGGC-3′ | reverse |

and partial sequences thereof and sequences that are hybridisable therewith preferably at a temperature of at least 20° C. and especially at a concentration of 1M NaCl and a temperature of at least 25° C., for the diagnosis of tuberculosis and other mycobacterial infections in humans and animals.

10. The use of a DNA sequence according to claim 9 for the diagnosis of tuberculosis and other mycobacterial infections in humans and animals.

11. A method according to claim 10, characterised in that a DNA sequence according to claim 9 is used
(i) for hybridisation,
(ii) for culture confirmation of isolated strains and/or
(iii) for chromosomal fingerprinting, and cells, strains and/or types of mycobacteria are determined and differentiated and/or are used for the diagnosis of mycobacterial infections.

12. A method according to claim 10 or 11, characterised in that cells, strains and/or species of virulent mycobacteria are differentiated from non-virulent cells, strains and/or species.

13. A method according to claim 10, characterised in that cells, strains and/or species of the M. tuberculosis complex and other mycobacteria
(i) are isolated,
(ii) crude or purified genomic DNA or RNA is recovered,
(iii) a fragment that is identical or virtually identical to the sequence of the alanine dehydrogenase gene of M. tuberculosis (FIG. 2.3) is identified, preferably by amplification using a DNA sequence according to claim 9 as a primer sequence, after which digestion is carried out with a restriction enzyme, especially Bg1II, and gel electrophoresis of the digested amplified DNA is carried out and/or the DNA sequence of the amplified DNA is determined.

14. A method according to claim 2 and/or 10, characterised in that a clinical sample is used directly and diagnosed for tuberculosis in humans and animals.

15. A method according to claim 2 and/or 10, characterised in that the method is carried out in the presence of substances that inhibit tuberculosis or mycobacterial infections of humans and animals and inhibiting substances determined are recovered or made.

16. A method according to claim 10, characterised in that it is used
(i) in antimycobacterial chemotherapy,
(ii) in the control of epidemics and/or
(iii) after vaccinations (vaccination follow-up) in humans and animals.
